(19) United States
(12) Patent Application Publication
(10) Pub. No.: US 2021/0307285 A1
(43) Pub. Date: Oct. 7, 2021

(54) SESAME PLANTS WITH IMPROVED ORGANOLEPTIC PROPERTIES AND METHODS THEREOF

(71) Applicant: Sabra Dipping Company, LLC, Colonial Heights, VA (US)

(72) Inventors: Meiky TOLLMAN, Colonial Heights, VA (US); Mario VAZQUEZ, Colonial Heights, VA (US); Oron GAR, M.P. Lachish Darom (IL); Arie ZACKAY, Jerusalem (IL); Gil SHALEV, Ramot Mehir (IL); Jerry BELTON, Colonial Heights, VA (US)

(21) Appl. No.: 17/212,682

(22) Filed: Mar. 25, 2021

Related U.S. Application Data
(60) Provisional application No. 63/004,718, filed on Apr. 3, 2020.

(51) Int. Cl. A01H 6/66; A01H 5/10; A23L 25/00; A23L 27/10

(52) U.S. Cl. CPC A01H 6/66; A01H 5/10; A23V 2002/00; A23L 27/10; A23L 25/30

(57) ABSTRACT

The invention relates to Sesamum indicum (sesame) plants comprising quantitative trait loci (QTL) associated with shatter resistant capsules and improved organoleptic properties and having a protein content of about 18% to about 25% by weight, a fat content of about 48% to about 56% by weight, and/or an L value of greater than 60 as measured, for example, by Hunter Colorflex color meter. Provided are sesame plants and seeds having these characteristics (both open pollinated and hybrids) as well as methods for breeding sesame plants, growing sesame plants, and food products made with the sesame plants and parts thereof, preferably the sesame seeds.

### CROSS-REFERENCE TO RELATED PATENT APPLICATION

This application claims priority from U.S. Provisional Patent Application No. 63/004,718, filed on Apr. 3, 2020, the entire disclosure of which is hereby incorporated by reference in its entirety.

### FIELD OF THE INVENTION

The invention relates to Sesamum indicum (sesame) plants having desired protein contents, fat contents and/or color in its seeds and comprising quantitative trait loci (QTL) associated with shatter resistant capsules and/or improved organoleptic properties. Provided are sesame plants and seeds having these characteristics (both open pollinated and hybrids) as well as methods for breeding sesame plants, growing sesame plants, and food products such as tahini made with the sesame plants and parts thereof, preferably the sesame seeds.

### BACKGROUND OF THE INVENTION

Sesame (Sesamum indicum) is an annual plant of pedaliaceae family, grown widely in tropical and subtropical areas and has a small (˜354 MB) diploid (2n=26) genome. Sesame is considered to be one of the important and oldest of the oilseed plants as it has been under cultivation in Asia for over 5000 years.

Sesame is an annual broadleaf plant that grows 5-6 ft (155-185 cm) tall. It produces a 1-2 in (2.5-5 cm) long white, bellshaped inflorescence growing from the leaf axils (where the leaf stalk joins the stem). The blooms do not open all at once, but gradually, from the base of the stem upwards to the top of the plant. The flowers are both male and female and will self-pollinate. The seed is produced in a 1-1.5 in (2.5-3.8 cm) long, divided seed capsule that opens when the seeds are mature. There are 8 rows of seed within each seed capsule. Seed capsules are 1 to 1½ inches long, with 8 rows of seeds in each capsule. Some varieties are branched, while others are unbranched. Sesame varieties have single or multiple stems.

Due to the nonuniform, indeterminate nature of the bloom period, the reproductive, ripening, and drying phases of the seed tend to overlap. Seed lowest on the plant will mature first, even as the upper part of the plant is still flowering or has just formed seed capsules. Since the flowering occurs in an indeterminate fashion, seed capsules on the lower stem are ripening while the upper stem is still flowering. The lowest flowers on a stem may not develop into pods, but pods will generally begin 12 to 24 inches off the ground and continue to the top of the stem.

Sesame seeds are small in size, and they occur in many colors depending on the cultivar. The most traded variety of sesame is off-white colored. Other common colors are buff, tan, gold, brown, reddish, gray, and black. The color is the same for the hull and the fruit. Form, and colors vary between the thousands of cultivated varieties. USDA Natural Resources Conservation Service Plant Guide—Sesame (2014); Iowa State University “Sesame” (2002).

Due to its shattering capsules, sesame seed crops must be harvested manually to prevent losing the seeds and due to this characteristic require intensive manual labor. Also, sesame seed organoleptic properties and seed color vary greatly and are inconsistent. Accordingly, there is a need in the art for sesame seeds that can be readily harvested by machine with consistent desirable organoleptic properties.

### SUMMARY OF VARIOUS EMBODIMENTS OF THE INVENTION

In an embodiment, the invention provides for a sesame plant or part thereof comprising introgressed organoleptic property loci associated with a plurality of quantitative trait loci (QTLs) associated with organoleptic properties, wherein said plurality of QTLs comprise S1, S2, S3, or a combination thereof, comprising introgressed shatter resistant capsule loci associated with a plurality of quantitative trait loci (QTLs) associated with shatter resistant capsules, wherein said plurality of QTLs associated with shatter resistant capsules comprise at least one of QTL 1, 2, 3, 4, 5, 6, 7, or a combination thereof, and having a protein content of about 18% to about 25% by weight, a fat content of about 48% to about 56% by weight, and/or an L value of greater than 60 as measured, for example, by Hunter Colorflex color meter in its seeds.

In an embodiment, the invention provides for a hybrid sesame plant obtained by crossing a plant grown from seeds of the sesame plant described herein, with another sesame plant. In an embodiment, the plant may comprise Marker Cassette S, wherein said Marker Cassette S may comprise LG6_19788548, LG6_6028959, LG8_18013656, or a combination thereof, wherein the alleles at the single nucleotide polymorphism (SNP) for said LG6_19788548, LG6_6028959, and LG8_18013656 are homozygous or heterozygous; and wherein the nucleic acid sequence of LG6_19788548 is set forth in SEQ ID NO: 17 or 18; wherein the nucleic acid sequence of LG6_6028959 is set forth in SEQ ID NO: 19 or 20; and wherein the nucleic acid sequence of LG8_18013656 is set forth in SEQ ID NO: 21 or 22.

In an embodiment, the Marker Cassette S may comprise LG6_19788548, LG6_6028959, and LG8_18013656. LG6_19788548, LG6_6028959, and LG8_18013656 may be homozygous. The nucleic acid sequence of LG6_19788548 may be set forth in SEQ ID NO: 17. The nucleic acid sequence of LG6_6028959 may be set forth in SEQ ID NO: 19. The nucleic acid sequence of LG8_18013656 may be set forth in SEQ ID NO: 21.

In an embodiment, the sesame plant described herein may comprise Marker Cassette 1, 2, 3, 4, (See Table 1) or a combination thereof, wherein said Marker Cassette 1 may comprise Reference or alternative alleles of LG3_19205572, LG7_5141423, LG15_5315334, or a combination thereof, wherein the alleles for said LG3_19205572, LG7_5141423, LG15_5315334 are homozygous or heterozygous; wherein said Marker Cassette 2 may comprise Reference or alternative alleles of LG3_19205572, LG11_8864255, LG15_5315334, or a combination thereof, wherein the alleles for said LG3_19205572, LG11_8864255, LG15_5315334 are homozygous or heterozygous; wherein said Marker Cassette 3 may comprise Reference or alternative alleles of LG3_19205572, LG5_12832234, LG15_4900868, LG15_5315334, or a combination thereof, wherein the alleles for said LG3_19205572, LG5_12832234, LG15_4900868, LG15_5315334 are homozygous or heterozygous; wherein said Marker Cassette 4 may comprise Reference or alternative alleles of LG6_2739268, LG11_8864255, LG16_1563304, or a combination thereof, wherein the alleles for said LG6_2739268, LG11_8864255, LG16_1563304 are homozygous or heterozygous; and wherein the nucleic acid sequence of LG3_19205572 may be set forth in SEQ ID NO: 1 or 9, wherein the nucleic acid sequence of LG5_12832234 may be set forth in SEQ ID NO: 2 or 10, wherein the nucleic acid sequence of LG6_2739268 may be set forth in SEQ ID NO: 3 or 11, wherein the nucleic acid sequence of LG7_5141423 may be set forth in SEQ ID NO: 4 or 12, wherein the nucleic acid sequence of LG11_8864255 may be set forth in SEQ ID NO: 5 or 13, wherein the nucleic acid sequence of LG15_4900868 may be set forth in SEQ ID NO: 6 or 14, wherein the nucleic acid sequence of LG15_5315334 may be set forth in SEQ ID NO: 7 or 15, and wherein the nucleic acid sequence of LG16_1563304 may be set forth in SEQ ID NO: 8 or 16.

In an embodiment, the nucleic acid sequence of LG3_19205572 may be set forth in SEQ ID NO: 1, wherein the nucleic acid sequence of LG5_12832234 may be set forth in SEQ ID NO: 2, wherein the nucleic acid sequence of LG6_2739268 may be set forth in SEQ ID NO: 11, wherein the nucleic acid sequence of LG7_5141423 may be set forth in SEQ ID NO: 4, wherein the nucleic acid sequence of LG11_8864255 may be set forth in SEQ ID NO: 5, wherein the nucleic acid sequence of LG15_4900868 may be set forth in SEQ ID NO: 14, wherein the nucleic acid sequence of LG15_5315334 may be set forth in SEQ ID NO: 15, wherein the nucleic acid sequence of LG16_1563304 may be set forth in SEQ ID NO: 16, or a combination thereof.

In an embodiment, the sesame plant described herein may have shatter resistant pods.

In an embodiment, the sesame plant described herein may have about 18% to about 25% protein content in its seeds. The sesame plant described herein may have about 19% or more or about 20% or more protein content in its seeds. The sesame plant described herein may have about 24% or less protein content in its seeds. Preferably, the sesame plant described herein may have about 20% to about 24% protein content in its seeds. The sesame plant described herein may have about 22% protein content in its seeds. Unless otherwise indicated, the content percentages in this disclosure refer to percentages by weight.

The sesame plant described herein may have about 48% to about 56% fat content in its seeds. The sesame plant described herein may have about 49% or more, or about 50% or more fat content in its seeds. The sesame plant described herein may have about 55% or less, or about 54% or less, fat content in its seeds. Preferably, the sesame plant described herein may have about 50% to 54% fat content in its seeds. The sesame plant described herein may have about 52% fat content in its seeds.

In an embodiment, the sesame plant described herein may produce sesame seeds that are whitish in appearance. The sesame plant described herein may produce sesame seeds having an L value greater than 60 as measured, for example, by Hunter Colorflex color meter. Preferably the L value is greater than about 63 as measured, for example, by Hunter Colorflex color meter.

In an embodiment, the sesame plant described herein may have about 15% carbohydrate content in its seeds. The sesame plant may have about 10-20% carbohydrate content in its seeds.

In an embodiment, the sesame plant described herein may have 1, 2, or 3 pods per node. The sesame plant may have 1 pods per node. The sesame plant may have 2 pods per node. The sesame plant may have 3 pods per node.

In an embodiment, the sesame plant described herein may have between 60 and 240, more preferably 180 to 240 capsules in its main branch. The sesame plant may have from 3 to 5, typically an average of 5 lateral branches. The sesame plant may have between 200 and 800, more preferably between 400 and 600 total capsules per plant. The sesame plant may show an initial flowering at about 15-85 cm above ground, preferably about 15 cm above the ground.

In an embodiment, the sesame plant described herein may be a variety.

In an embodiment, the invention provides for an isolated plant cell of the sesame plant described herein.

In an embodiment, the invention provides for a sesame plant grown from the seed of the sesame plant described herein.

In an embodiment, the invention provides for a part of the sesame plant described herein. The part may be seed, seed fragment, an endosperm, plant cell, cell culture, a tissue culture, a protoplast, pollen, an ovule, a meristem, an embryo, or a plant organ. The plant part may be a capsule. The plant part may be a seed. The plant part may be a seed fragment.

In an embodiment, the invention provides for a tissue culture of cells obtained from the sesame plant described herein, wherein said tissue culture of cells is from a tissue from the leaf, pollen, embryo, bulb, anther, flower, bud, or meristem.

In an embodiment, the invention provides for a container comprising a plurality of the sesame plant or part thereof described herein. The container may be a bag, can, packet, box, cargo tote, or flat. The container may contain capsules. The container may contain sesame seeds. The container may contain defatted sesame seeds. The container may contain sesame seed fragments. At least 10%, or at least 20%, or at least 30% of the sesame seed or sesame plant parts in the container may be derived from sesame plants of this invention.

In an embodiment, the invention provides for a food product comprising the sesame plant or part thereof described herein. Preferably, the food product may comprise a paste comprising sesame seeds. The paste may be a tahini. The food product may be a dip comprising tahini made from sesame seeds of the sesame seed plant described herein. The dip may be hummus, or baba ganoush. The food product may be ice cream comprising tahini made from sesame seeds of the sesame seed plant described herein.

In an embodiment, the invention provides for a tahini comprising sesame seeds comprising introgressed organoleptic property loci associated with a plurality of quantitative trait loci (QTLs) associated with organoleptic properties, wherein said plurality of QTLs comprise S1, S2, S3, or a combination thereof, comprising introgressed shatter resistant capsule loci associated with a plurality of quantitative trait loci (QTLs) associated with shatter resistant capsules, wherein said plurality of QTLs associated with shatter resistant capsules comprise at least one of QTL 1, 2, 3, 4, 5, 6, 7, or a combination thereof, and the sesame seeds having a protein content of about 18% to about 25% by weight, a fat content of about 48% to about 56% by weight, and/or an L value of greater than 60 as measured, for example, by Hunter Colorflex color meter. At least about 10% of the sesame-derived material in the tahini will be derived from sesame plants of this invention to improve the flavor of the tahini. Preferably, at least about 20%, and more preferably, at least about 30% of the sesame-derived material in the tahini may be derived from sesame plants of this invention.

In an embodiment, the invention provides for a method of making a food product such as tahini, comprising: selecting sesame seeds having about 18% to about 25% protein content by weight, about 48% to about 56% fat content by weight, and/or an L value of greater than 60, as measured, for example, by Hunter Colorflex color meter; and admixing the sesame seeds with ingredients to produce the food product. Preferably, the sesame seeds have about 20% to about 24% protein content by weight, about 50% to about 54% fat content by weight, and/or an L value of greater than 63, as measured, for example, by Hunter Colorflex color meter.

In one embodiment, the method of making a food product such as tahini comprises admixing the sesame plant part described herein with ingredients to produce the food product. The sesame plant part such as sesame seeds may comprise introgressed organoleptic property loci associated with a plurality of quantitative trait loci (QTLs) associated with organoleptic properties, wherein said plurality of QTLs comprise S1, S2, S3, or a combination thereof, may comprise introgressed shatter resistant capsule loci associated with a plurality of quantitative trait loci (QTLs) associated with shatter resistant capsules, wherein said plurality of QTLs associated with shatter resistant capsules comprise at least one of QTL 1, 2, 3, 4, 5, 6, 7, or a combination thereof, and may have a protein content of about 18% to about 25% by weight, a fat content of about 48% to about 56% by weight, and/or an L value of greater than 60 as measured, for example, by Hunter Colorflex color meter. The method may further comprise roasting the sesame seeds. In one embodiment, the method of making a food product may comprise comminuting the sesame seeds of the sesame plant described herein.

In one embodiment, the method of making tahini may comprise roasting and comminuting the sesame seed of the sesame plant described herein. The sesame seeds may be roasted before comminuting. The sesame seeds may be comminuted and then roasted. The method may further comprise cleaning said sesame seeds, washing, drying, dehulling, roasting, and comminuting said sesame seeds.

In one embodiment, a method of producing a hybrid sesame seed may comprise crossing the sesame plant described herein with another sesame plant; and obtaining F1 sesame plant.

In one embodiment, a method for producing sesame plants or seeds may comprise growing a sesame plant from the F1 seeds the sesame plant described herein, crossing the F1 sesame plant with a sesame plant, and obtaining F2 seeds from said cross.

In one embodiment, a method of producing sesame seeds may comprise growing the sesame plant described herein and harvesting the sesame capsules and/or seeds. The harvesting may be done by machine.

In one embodiment, the invention provides for a field comprising the sesame plant described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows shatter resistant capsule QTL 1-7 and organoleptic QTL S1-S3 and linked markers on Sesamum indicum linkage groups. The circles with numbers represent a marker combination set (“cassette”).

FIG. 2A-B depicts two sesame plants comprising QTL1-7 and QTL S1, S2, and S3, and a child plant comprising QTL1-7 and QTL S1, S2, and S3. Destiny Type Line A (FIG. 2A) and Destiny Type Line B (FIG. 2B).

FIG. 3A-C shows the DNA sequence corresponding to QTL S1 (LG6-19788548, SEQ ID NO: 17 and SEQ ID NO: 18) (FIG. 3A); QTL S2 (LG6-6028959, SEQ ID NO: 19 and SEQ ID NO: 20) (FIG. 3B); and, QTL S3 (LG8-18013656, SEQ ID NO: 21 and SEQ ID NO: 22) (FIG. 3C).

FIG. 4 shows the relationship between seed color as indicated by L value and the protein content of the sesame seeds in Example 4 and the relationship between the fat content and the protein content of the sesame seeds in Example 4.

FIG. 5 shows the JMP partition decision tree for 25 sesame varieties having protein contents averaging 20 to 24% in 2018 crop year.

### DETAILED DESCRIPTION OF PREFERRED EMBODIMENTS

### Definitions

Unless otherwise indicated, all terms used herein have the same meaning as they would to one skilled in the art.

In the present disclosure the singular forms “a,” “an,” and “the” include the plural reference, and reference to a particular numerical value includes at least that particular value, unless the context clearly indicates otherwise. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

“Associated with” or “associated,” as used herein, refers broadly to a nucleic acid and a phenotypic trait, that are in linkage disequilibrium. For example, the nucleic acid and the trait are found together in progeny plants more often than if the nucleic acid and phenotype segregated separately.

“Crossed” or “cross” in the context of this invention means the fusion of gametes via pollination to produce progeny (e.g., cells, seeds, or plants). The term encompasses both sexual crosses (the pollination of one plant by another) and selfing (self-pollination, e.g., when the pollen and ovule are from the same plant).

“Dicot,” as used herein, refers broadly to the subclass of angiosperm plants also knows as “dicotyledoneae” and includes reference to whole plants, plant organs (e.g., leaves, stems, roots), seeds, plant cells, and progeny of the same. Plant cell, as used herein includes, without limitation, seeds, suspension cultures, embryos, meristematic regions, callus tissue, leaves, roots, shoots, gametophytes, sporophytes, pollen, and microspores.

“Food product,” as used herein, refers broadly to any substance that can be used or prepared for use as food. Food product, as used herein, includes ingredients used to make food products, e.g., tahini. Food product, as used herein, also includes animal feed made from the claimed sesame seed plant and byproducts thereof.

“Interval,” as used herein, refers broadly to a continuous linear span of chromosomal DNA with termini defined by and including molecular markers.

“Linkage disequilibrium,” as used herein, refers broadly to a non-random segregation of genetic loci. This implies that such loci are in sufficient physical proximity along a length of a chromosome that they tend to segregate together with greater than random frequency.

“Marker” or “molecular marker,” as used herein, refers broadly to a genetic locus (a “marker locus”) used as a point of reference when identifying genetically linked loci such as a QTL. The term also refers to nucleic acid sequences complementary to the genomic sequences, such as nucleic acids used as probes.

“Nucleic acid,” “polynucleotide,” “polynucleotide sequence” and “nucleic acid sequence,” as used herein, refers broadly to single-stranded or double-stranded deoxyribonucleotide or ribonucleotide polymers, or chimeras thereof. As used herein, the term can additionally or alternatively include analogs of naturally occurring nucleotides having the essential nature of natural nucleotides in that they hybridize to single-stranded nucleic acids in a manner similar to naturally occurring nucleotides (e.g., peptide nucleic acids). Unless otherwise indicated, a particular nucleic acid sequence of this invention optionally encompasses complementary sequences, in addition to the sequence explicitly indicated. The term “gene” is used to refer to, e.g., a cDNA and an mRNA encoded by the genomic sequence, as well as to that genomic sequence.

“Genetically linked,” as used herein, refers broadly to genetic loci that are in linkage disequilibrium and statistically determined not to assort independently. Genetically linked loci assort dependently from 51% to 99% of the time or any whole number value therebetween, preferably at least 60%, 70%, 80%, 90%, 95% or 99%.

“Genotype,” as used herein, refers broadly to the total of inheritable genetic information of a plant, partly influenced by the environmental factors, which is expressed in the phenotype.

“Homologous,” as used herein, refers broadly to nucleic acid sequences that are derived from a common ancestral gene through natural or artificial processes (e.g., are members of the same gene family), and thus, typically, share sequence similarity. Typically, homologous nucleic acids have sufficient sequence identity that one of the sequences or its complement is able to selectively hybridize to the other under selective hybridization conditions. The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences have about at least 80% sequence identity, preferably at least 90% sequence identity, and most preferably 95%, 97%, 99%, or 100% sequence identity with each other. A nucleic acid that exhibits at least some degree of homology to a reference nucleic acid can be unique or identical to the reference nucleic acid or its complementary sequence.

“Host cell,” as used herein, refers broadly to a cell which contains a heterologous nucleic acid, such as a vector, and supports the replication and/or expression of the nucleic acid. Host cells may be prokaryotic cells such as E. coli, or eukaryotic cells such as yeast, insect, amphibian, or mammalian cells. Preferably, host cells are monocotyledonous or dicotyledonous plant cells. In the context of the invention, the host cell may be a soybean host cell or a sesame seed host cell.

“Hybrid” or “hybrid plant,” as used herein, refers broadly to a plant produced by the inter-crossing (cross-fertilization) of at least two different plants or plants of different parent lines. The seeds of such a cross (hybrid seeds) are encompassed, as well as the hybrid plants grown from those seeds and plant parts derived from those grown plants (e.g., seeds).

“F1, F2, seq al.,” as used herein, refers broadly to the consecutive related generations following a cross between two parent plants or parent lines. The plants grown from the seeds produced by crossing two plants or lines is called the F1 generation. Selfing the F1 plants results in the F2 generation.

“Introduced,” as used herein, refers broadly to a heterologous or isolated nucleic acid refers to the incorporation of a nucleic acid into a eukaryotic or prokaryotic cell where the nucleic acid can be incorporated into the genome of the cell (e.g., chromosome, plasmid, plastid or mitochondrial DNA), converted into an autonomous replicon, or transiently expressed (e.g., transfected mRNA). The term includes such nucleic acid introduction means as “transfection,” “transformation” and “transduction.”

“Introgression,” as used herein, refers broadly to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene or a selected allele of a marker or QTL.

“Isolated,” as used herein, refers broadly to material, such as a nucleic acid or a protein, which is substantially free from components that normally accompany or interact with it in its naturally occurring environment. The isolated material optionally comprises material not found with the material in its natural environment, e.g., a cell. In addition, if the material is in its natural environment, such as a cell, the material has been placed at a location in the cell (e.g., genome or subcellular organelle) not native to a material found in that environment. For example, a naturally occurring nucleic acid (e.g., a promoter) is considered to be isolated if it is introduced by non-naturally occurring means to a locus of the genome not native to that nucleic acid. Nucleic acids which are “isolated” as defined herein, are also referred to as “heterologous” nucleic acids.

“Marker cassette,” as used herein refers broadly to a set of multiple genetic loci (QTL) associated with a desired phenotypic trait. The various genetic loci of the marker cassette are not necessarily genetically-linked, but particular alleles of the respective loci are consistently found in the genomes of plants with the same phenotypic trait.

“Phenotype,” as used herein, refers broadly to the observable external and/or physiological appearance of the plant as a result of the interaction between its genotype and its environment. It includes all observable morphological and physiological characteristics.

“Plant,” as used herein, refers broadly to the whole plant or any parts or derivatives thereof, such as plant organs (e.g., harvested or non-harvested storage organs, bulbs, tubers, fruits, leaves), plant cells, plant protoplasts, plant cell tissue cultures from which whole plants can be regenerated, plant calli, plant cell clumps, and plant cells that are intact in plants, or parts of plants, such as embryos, pollen, ovules, fruits (e.g., capsule, harvested tissues or organs), flowers, leaves, seeds, seed fragments (e.g., milled sesame seeds), tubers, bulbs, clonally propagated plants, roots, stems, root tips. Also any developmental stage is included, such as seedlings, immature and mature bulbs.

“Proximal,” as used herein, refers broadly genetically linked loci, including alleles, usually within about 1-30 centiMorgans (cM).

“Quantitative trait locus” or “QTL,” as used herein, refers broadly to a polymorphic genetic locus with at least two alleles that differentially affect the expression of a continuously distributed phenotypic trait. Further, a quantitative trait locus (QTL) may broadly refer to a locus (i.e., section of DNA) which correlates with variation of a quantitative trait in the phenotype of a population of organisms. QTLs may be identified using molecular markers, such as SNPs or AFLPs, that correlate with an observed phenotypic trait.

“Seed,” as used herein refers broadly the ripened ovule of a flowering plant containing an embryo and capable normally of germination to produce a new plant.

“Selfing” in the context of this invention means self-pollination (e.g., when the pollen and ovule are from the same plant).

“Variety,” as used herein, refers broadly to a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged. See, e.g., USDA definitions.

Quantitative Trait Loci (QTL) Associated with Shatter Resistant Capsule Phenotype

The QTLs of the invention associated with a shatter resistant capsule phenotype comprise one or more of QTLs 1 to 7. In one embodiment, the alleles of one or more markers linked to QTLs 1-7 are homozygous. In another embodiment, the alleles of one or more markers linked to QTLs 1-7 are heterozygous. QTLs 1-7 are associated with the shatter resistant capsule phenotype such that the sesame plant comprising QTLs 1-7 in its genome can be harvested by machine. A more complete description of QTLs 1-7, their discovery, and sesame plants exhibiting the shatter resistant capsule genotype and phenotype is provided in U.S. Published Application 2018/0355368, which is incorporated herein by reference.

“QTL 1,” as used herein refers to a polymorphic genetic locus linked to genetic marker LG3_19205572 in sesame linkage group 3. In one embodiment, the alleles of LG3_19205572 are homozygous. In another embodiment, the alleles of LG3_19205572 are heterozygous. In one embodiment, a first allele of LG3_19205572 may have the base ‘C’ at position 19205572, and a second allele may have the base ‘T’ instead of ‘C’ at position 19205572. The nucleic acid sequence of the first allele of LG3_19205572 marker is set forth in SEQ ID NO: 1, and the nucleic acid sequence of the second allele of LG3_19205572 marker is set forth in SEQ ID NO: 9. All sequences described herein are from Sesame genome version 1. See Wang et al. (2014) Genome Biology 15(2): R39.

“QTL 2,” as used herein refers to a polymorphic genetic locus linked to genetic marker LG5_12832234 in sesame linkage group 5. In one embodiment, the alleles of LG5_12832234 are homozygous. In another embodiment, the alleles of LG5_12832234 are heterozygous. In one embodiment, a first allele of LG5_12832234 may have the base ‘C’ at position 12832234, and a second allele may have the base ‘T’ instead of ‘C’ at position 12832234. The nucleic acid sequence of the first allele of LG5_12832234 marker is set forth in SEQ ID NO: 2, and the nucleic acid sequence of the second allele of LG5_12832234 marker is set forth in SEQ ID NO: 10.

“QTL 3,” as used herein refers to a polymorphic genetic locus linked to genetic marker LG6_2739268 in sesame linkage group 6. In one embodiment, the alleles of LG6_2739268 are homozygous. In another embodiment, the alleles LG6_2739268 are heterozygous. In one embodiment, a first allele of LG6_2739268 may have the base ‘T’ at position 2739268, and a second allele may have the base ‘C’ instead of ‘T’ at position 2739268. The nucleic acid sequence of the first allele of LG6_2739268 marker is set forth in SEQ ID NO: 3, and the nucleic acid sequence of the second allele of LG6_2739268 marker is set forth in SEQ ID NO: 11.

“QTL 4,” as used herein refers to a polymorphic genetic locus linked to genetic marker LG7_5141423 in sesame linkage group 7. In one embodiment, the alleles of LG7_5141423 are homozygous. In another embodiment, the alleles LG7_5141423 are heterozygous. In one embodiment, a first allele of LG7_5141423 may have the base ‘C’ at position 5141423, and a second allele may have the base ‘G’ instead of ‘C’ at position 5141423. The nucleic acid sequence of the first allele of LG7_5141423 marker is set forth in SEQ ID NO: 4, and the nucleic acid sequence of the second allele of LG7_5141423 marker is set forth in SEQ ID NO: 12.

“QTL 5,” as used herein refers to a polymorphic genetic locus linked to genetic marker LG11_8864255 in sesame linkage group 11. In one embodiment, the alleles of LG11_8864255 are homozygous. In another embodiment, the alleles LG11_8864255 are heterozygous. In one embodiment, a first allele of LG11_8864255 may have the base ‘C’ at position 8864255, and a second allele may have the base G′ instead of ‘C’ at position 8864255. The nucleic acid sequence of the first allele of LG11_8864255 marker is set forth in SEQ ID NO: 5, and the nucleic acid sequence of the second allele of LG11_8864255 marker is set forth in SEQ ID NO: 13.

“QTL 6,” as used herein refers to a polymorphic genetic locus linked to genetic markers LG15_4900868 and LG15_5315334 in sesame linkage group 15. In one embodiment, the alleles of LG15_4900868 are homozygous. In another embodiment, the alleles LG15_4900868 are heterozygous. In one embodiment, a first allele of LG15_4900868 may have the base G′ at position 4900868, and a second allele may have the base ‘A’ instead of G′ at position 4900868. In one embodiment, the alleles of LG15_5315334 are homozygous. In another embodiment, the alleles LG15_5315334 are heterozygous. In one embodiment, a first allele of LG15_5315334 may have the base ‘T’ at position 5315334, and a second allele may have the base ‘C’ instead of ‘T’ at position 5315334. The nucleic acid sequence of the first allele of LG15_4900868 marker is set forth in SEQ ID NO: 6, the nucleic acid sequence of the second allele of LG15_4900868 marker is set forth in SEQ ID NO: 14, the nucleic acid sequence of the first allele of LG15_5315334 marker is set forth in SEQ ID NO: 7, and the nucleic acid sequence of the second allele of LG15_5315334 marker is set forth in SEQ ID NO: 15.

“QTL 7,” as used herein refers to a polymorphic genetic locus linked to genetic marker LG16_1563304 in sesame linkage group 16. In one embodiment, the alleles of LG16_1563304 are homozygous. In another embodiment, the alleles LG16_1563304 are heterozygous. In one embodiment, a first allele of LG16_1563304 may have the base ‘A’ at position 1563304, and a second allele may have the base G′ instead of ‘A’ at position 1563304. The nucleic acid sequence of the first allele of LG16_1563304 marker is set forth in SEQ ID NO: 8, and the nucleic acid sequence of the second allele of LG16_1563304 marker is set forth in SEQ ID NO: 16.

Quantitative Trait Loci (QTL) Associated with Organoleptic Properties

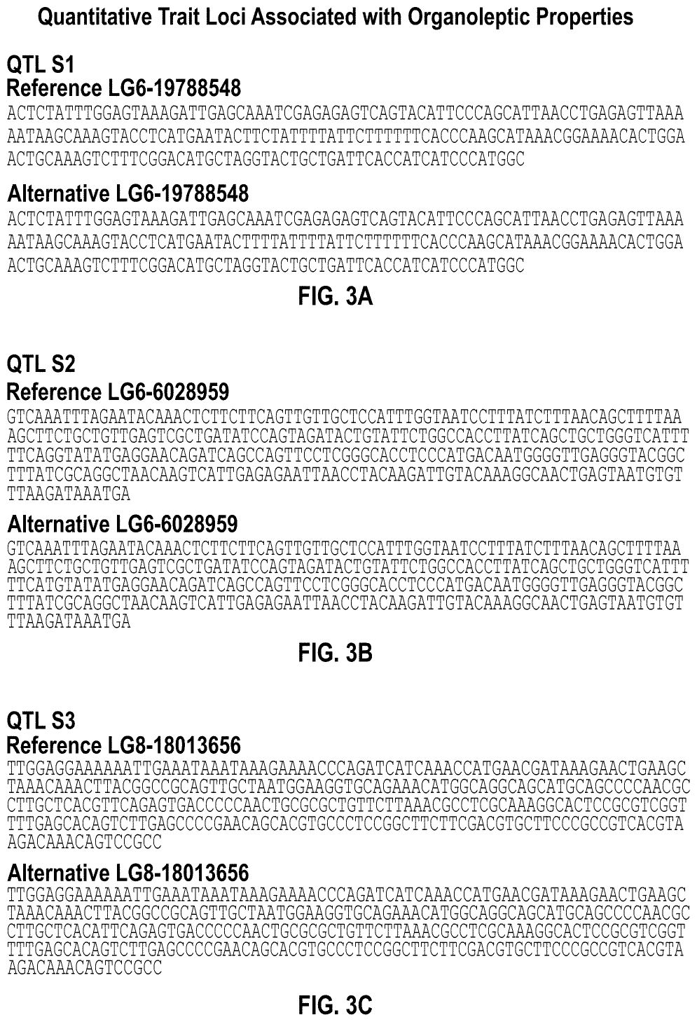

The marker cassettes of the invention associated with desired organoleptic properties comprise one or more of QTLs S1, S2, and S3 (See FIG. 3). In one embodiment, the alleles of one or more markers linked to QTLs S1, S2, and S3 are homozygous. In another embodiment, the alleles of one or more markers linked to QTLs S1, S2, and S3 are heterozygous.

“QTL S1,” as used herein refers to a polymorphic genetic locus linked to genetic marker LG6_19788548 in sesame linkage group 6. In one embodiment, the alleles of LG6_19788548 are homozygous. In another embodiment, the alleles LG6_19788548 are heterozygous. In one embodiment, a first allele of LG6_19788548 may have the base ‘C’ at position 19788548, and a second allele may have the base ‘T’ instead of ‘C’ at position 19788548. The nucleic acid sequence of the first allele of LG6_19788548 marker is set forth in SEQ ID NO: 17, and the nucleic acid sequence of the second allele of LG6_19788548 marker is set forth in SEQ ID NO: 18.

“QTL S2,” as used herein refers to a polymorphic genetic locus linked to genetic marker LG6_6028959 in sesame linkage group 6. In one embodiment, the alleles of LG6_6028959 are homozygous. In another embodiment, the alleles LG6_6028959 are heterozygous. In one embodiment, a first allele of LG6_6028959 may have the base ‘G’ at position 6028959, and a second allele may have the base ‘T’ instead of G′ at position 6028959. The nucleic acid sequence of the first allele of LG6_6028959 marker is set forth in SEQ ID NO: 19, and the nucleic acid sequence of the second allele of LG6_6028959 marker is set forth in SEQ ID NO: 20.

“QTL S3,” as used herein refers to a polymorphic genetic locus linked to genetic marker LG8_18013656 in sesame linkage group 8. In one embodiment, the alleles of LG8_18013656 are homozygous. In another embodiment, the alleles LG8_18013656 are heterozygous. In one embodiment, a first allele of LG8_18013656 may have the base ‘G’ at position 18013656, and a second allele may have the base ‘A’ instead of ‘G’ at position 18013656. The nucleic acid sequence of the first allele of LG8_18013656 marker is set forth in SEQ ID NO: 21 and the nucleic acid sequence of the second allele of LG8_18013656 marker is set forth in SEQ ID NO: 22.

QTLs 1-7 are associated with the shatter resistant capsule phenotype such that the sesame plant comprising at least one, but preferably at least three of QTLs 1-7 in its genome can be harvested by machine. QTLs S1, S2, and S3 are associated with desired organoleptic properties and a white seed phenotype. In a preferred embodiment, the sesame seed harvested from a sesame seed plant comprising at least one, preferably at least two, or alternatively all three QTLs S1, S2, and S3 has a protein content of about 18% to about 25%, more preferably about 20% to about 24%, and a fat composition of about 48% to about 56%, more preferably about 50% to about 54%, in its seeds. Typical values for sesame seed according to this invention for carbohydrate are 9-26% and for ash are 3-8%.

In a preferred embodiment, the sesame seed harvested from a sesame seed plant comprising one or more of QTLs S1, S2, and/or S3 has a seed color that is whitish. The seeds may be off-white or white in color. The inventors developed a technique to measure seed color. The “Lab” color space is a color space defined by the International Commission on Illumination (CIE) in 1976. It expresses color as three numerical values, L* for the lightness and a* and b* for the green-red and blue-yellow color components. CIELAB was designed to be perceptually uniform with respect to human color vision, meaning that the same amount of numerical change in these values corresponds to about the same amount of visually perceived change. The lightness value, L*, represents the darkest black at L*=0, and the brightest white at L*=100. The color channels, a* and b*, represent true neutral gray values at a*=0 and b*=0. The a* axis represents the green-red component, with green in the negative direction and red in the positive direction. The b* axis represents the blue-yellow component, with blue in the negative direction and yellow in the positive direction. The seeds of the sesame plants described herein may have a seed with seed color values ranges of 60 to 85, or 65 to 85, preferably more than 71 for color L and a range of 0.75 to 5.5, preferably 4 to 5.5 for color A and 6-29, preferably 10 to 15 for color B making the seed whitish in appearance. In certain embodiments, the seeds of the sesame plants described herein may have an L value of greater than 60, preferably greater than 63, as measured, for example, by Hunter Colorflex color meter in its seeds.

One preferred embodiment includes sesame plants and/or plant parts which comprise Marker Cassette S which in turn comprise QTLs LG6_19788548, LG6_6028959, and LG8_18013656, or a combination thereof. Another preferred embodiment encompasses sesame plants and plant parts which comprise at least one of QTLs S1, S2, and S3, plus at least three of QTLs 1-7.

### Markers and Methods for Detection of Quantitative Trait Loci (QTL)

Suitable markers are genetically linked to the QTLs 1-7 identified herein as associated with shatter resistant capsules and genetically linked to the QTLs S1, S2, and S3 identified herein as associated with organoleptic properties and seed characteristics.

Markers can be identified by any of a variety of genetic or physical mapping techniques. Methods of determining whether markers are genetically linked to a QTL (or to a specified marker) associated with shatter resistant capsules and/or organoleptic properties are known in the art and include, for example, but not limited to, interval mapping (Lander and Botstein (1989) Genetics 121:185), regression mapping (Haley and Knott (1992) Heredity 69:315) or MQM mapping (Jansen (1994) Genetics 138:871). In addition, physical mapping techniques such as, for example, chromosome walking, contig mapping and assembly, and the like, can be employed to identify and isolate additional sequences useful as markers in the context of the present invention.

The markers may be homologous markers. Homologous markers can be identified by, for example, selective hybridization to a reference sequence. The reference sequence is typically a unique sequence, such as, for example, unique oligonucleotide primer sequences, ESTs, amplified fragments (e.g., corresponding to AFLP markers) and the like, derived from the marker loci of the invention.

In one example, the homologous markers hybridize with their complementary region. For example, two single-stranded nucleic acids “hybridize” when they form a double-stranded duplex. The double stranded region can include the full-length of one or both of the single-stranded nucleic acids, or all of one single stranded nucleic acid and a subsequence of the other single-stranded nucleic acid, or the double stranded region can include a subsequence of each nucleic acid. Selective hybridization conditions distinguish between nucleic acids that are related, e.g., share significant sequence identity with the reference sequence (or its complement) and those that associate with the reference sequence in a non-specific manner. Generally, selective hybridization conditions are described known in the art.

The methods for detecting genetic markers are described known in the art, for example, in U.S. Pat. Nos. 8,779,233; 6,670,524; 8,692,064; 9,000,258; 8,987,549; 8,637,729; 6,670,524; 6,455,758; 5,981,832; 5,492,547; 9,167,795; 8,656,692; 8,664,472; 8,993,835; 9,125,372; 9,144,220; 9,462,820; 7,250,552; and 9,485,936; and U.S. Patent Application Publications Nos. 2015/0082476; 2011/0154528; 2014/0215657; 2017/0055481; 2015/0150155; and 2015/0101073.

Markers corresponding to genetic polymorphisms between members of a population can be detected by numerous methods, described in the art, for example, but not limited to, restriction fragment length polymorphisms, isozyme markers, allele specific hybridization (ASH), amplified variable sequences of the plant genome, self-sustained sequence replication, simple sequence repeat (SSR), single nucleotide polymorphism (SNP), or amplified fragment length polymorphisms (AFLP).

The majority of genetic markers rely on one or more property of nucleic acids for their detection. For example, some techniques for detecting genetic markers utilize hybridization of a probe nucleic acid to nucleic acids corresponding to the genetic marker. Hybridization formats include, for example, but not limited to, solution phase, solid phase, mixed phase, or in situ hybridization assays. Markers which are restriction fragment length polymorphisms (RFLP), are detected by hybridizing a probe which is typically a sub-fragment (or a synthetic oligonucleotide corresponding to a sub-fragment) of the nucleic acid to be detected to restriction digested genomic DNA. The restriction enzyme is selected to provide restriction fragments of at least two alternative (or polymorphic) lengths in different individuals, and will often vary from line to line. Determining a (one or more) restriction enzyme that produces informative fragments for each cross is a simple procedure, described in the art. After separation by length in an appropriate matrix (e.g., agarose) and transfer to a membrane (e.g., nitrocellulose, nylon), the labeled probe is hybridized under conditions which result in equilibrium binding of the probe to the target followed by removal of excess probe by washing.

Nucleic acid probes to the marker loci can be cloned and/or synthesized. Detectable labels suitable for use with nucleic acid probes include, for example, but not limited to, any composition detectable by spectroscopic, radioisotopic, photochemical, biochemical, immunochemical, electrical, optical or chemical means. Useful labels include, for example, biotin for staining with labeled streptavidin conjugate, magnetic beads, fluorescent dyes, radiolabels, enzymes, and colorimetric labels. Other labels include ligands which bind to antibodies labeled with fluorophores, chemiluminescent agents, and enzymes. Labeling markers is readily achieved such as, for example, by the use of labeled PCR primers to marker loci.

The hybridized probe is then detected using any suitable technique known in the art, for example autoradiography or other similar detection technique (e.g., fluorography, liquid scintillation counter). Examples of specific hybridization protocols are described in the art.

Amplified variable sequences may refer to amplified sequences of the plant genome which exhibit high nucleic acid residue variability between members of the same species. Organisms have variable genomic sequences and each organism has a different set of variable sequences. Once identified, the presence of specific variable sequence can be used to predict phenotypic traits. Preferably, DNA from the plant serves as a template for amplification with primers that flank a variable sequence of DNA. The variable sequence is amplified and then sequenced.

In vitro amplification techniques are described in the art. Examples of techniques include, for example, but not limited to, the polymerase chain reaction (PCR), the ligase chain reaction (LCR), Q(3-replicase amplification and other RNA polymerase mediated techniques (e.g., NASBA), are found in Berger, Sambrook and Ausubel (all supra) as well as Mullis et al. (1987) U.S. Pat. No. 4,683,202; PCR Protocols, A Guide to Methods and Applications (Innis et al., Eds.) Academic Press Inc., San Diego Academic Press Inc. San Diego, Calif. (1990) (Innis); Arnheim & Levinson (1990) C&EN 36-47; The Journal Of NIH Research (1991) 3, 81-94; Kwoh et al. (1989) Proc. Natl. Acad. Sci. USA 86, 1173; Guatelli et al. (1990) Proc. Natl. Acad. Sci. USA 87, 1874; Lomeli et al. (1989) J. Clin. Chem. 35, 1826; Landegren et al., (1988) Science 241, 1077-1080; Van Brunt (1990) Biotechnology 8, 291-294; Wu & Wallace, (1989) Gene 4, 560; Barringer et al. (1990) Gene 89, 117, and Sooknanan & Malek (1995) Biotechnology 13: 563-564. Improved methods of cloning in vitro amplified nucleic acids are also described in U.S. Pat. No. 5,426,039. Improved methods of amplifying large nucleic acids by PCR can be found in Cheng et al. (1994) Nature 369: 684, and the references therein, in which PCR amplicons of up to 40 kb are generated.

Oligonucleotides for use as primers, e.g., in amplification reactions and for use as nucleic acid sequence probes are typically synthesized chemically according to, for example, the solid phase phosphoramidite triester method described by Beaucage and Caruthers (1981) Tetrahedron Lett. 22:1859.

Alternatively, self-sustained sequence replication can be used to identify genetic markers. Self-sustained sequence replication refers to a method of nucleic acid amplification using target nucleic acid sequences which are replicated exponentially in vitro under substantially isothermal conditions by using three enzymatic activities involved in retroviral replication: (1) reverse transcriptase, (2) RNAase H, and (3) a DNA-dependent RNA polymerase. Guatelli et al. (1990) Proc Natl Acad Sci USA 87:1874. By mimicking the retroviral strategy of RNA replication by means of cDNA intermediates, this reaction accumulates cDNA and RNA copies of the original target.

Amplified fragment length polymorphisms (AFLP) can also be used as genetic markers. Vos et al. (1995) Nucl Acids Res 23:4407. The phrase “amplified fragment length polymorphism” refers to selected restriction fragments which are amplified before or after cleavage by a restriction endonuclease. The amplification step allows easier detection of specific restriction fragments. AFLP allows the detection large numbers of polymorphic markers and has been used for genetic mapping of plants. Becker et al. (1995) Mol Gen Genet. 249:65; and Meksem et al. (1995) Mol Gen Genet. 249:74.

Allele-specific hybridization (ASH) can be used to identify the genetic markers of the invention. ASH technology is based on the stable annealing of a short, single-stranded, oligonucleotide probe to a completely complementary single-strand target nucleic acid. Detection is via an isotopic or non-isotopic label attached to the probe.

For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization. In this manner, only one of the alternative probes will hybridize to a target sample that is homozygous or homogenous for an allele. Samples that are heterozygous or heterogeneous for two alleles will hybridize to both of two alternative probes.

ASH markers are used as dominant markers where the presence or absence of only one allele is determined from hybridization or lack of hybridization by only one probe. The alternative allele may be inferred from the lack of hybridization. ASH probe and target molecules are optionally RNA or DNA; the target molecules are any length of nucleotides beyond the sequence that is complementary to the probe; the probe is designed to hybridize with either strand of a DNA target; the probe ranges in size to conform to variously stringent hybridization conditions, etc.

PCR allows the target sequence for ASH to be amplified from low concentrations of nucleic acid in relatively small volumes. Otherwise, the target sequence from genomic DNA is digested with a restriction endonuclease and size separated by gel electrophoresis. Hybridizations typically occur with the target sequence bound to the surface of a membrane or, as described, for example, in U.S. Pat. No. 5,468,613, the ASH probe sequence may be bound to a membrane.

ASH data can be obtained by amplifying nucleic acid fragments (amplicons) from genomic DNA using PCR, transferring the amplicon target DNA to a membrane in a dot-blot format, hybridizing a labeled oligonucleotide probe to the amplicon target, and observing the hybridization dots by autoradiography.

Single nucleotide polymorphisms (SNP) are markers that consist of a shared sequence differentiated on the basis of a single nucleotide. Typically, this distinction is detected by differential migration patterns of an amplicon comprising the SNP on e.g., an acrylamide gel. However, alternative modes of detection, such as hybridization, e.g., ASH, or RFLP analysis are not excluded.

In yet another basis for providing a genetic linkage map, Simple sequence repeats (SSR), take advantage of high levels of di-, tri-, or tetra-nucleotide tandem repeats within a genome. Dinucleotide repeats have been reported to occur in the human genome as many as 50,000 times with n varying from 10 to 60 or more. Jacob et al. (1991) Cell 67: 213. Dinucleotide repeats have also been found in higher plants. Condit & Hubbell (1991) Genome 34: 66.

Briefly, SSR data is generated by hybridizing primers to conserved regions of the plant genome which flank the SSR sequence. PCR is then used to amplify the dinucleotide repeats between the primers. The amplified sequences are then electorphoresed to determine the size and therefore the number of di-, tri-, and tetra-nucleotide repeats.

Alternatively, isozyme markers are employed as genetic markers. Isozymes are multiple forms of enzymes which differ from one another in their amino acid, and therefore their nucleic acid sequences. Some isozymes are multimeric enzymes containing slightly different subunits. Other isozymes are either multimeric or monomeric but have been cleaved from the proenzyme at different sites in the amino acid sequence. Isozymes can be characterized and analyzed at the protein level, or alternatively, isozymes which differ at the nucleic acid level can be determined. In such cases any of the nucleic acid based methods described herein can be used to analyze isozyme markers.

In alternative embodiments, in silico methods can be used to detect the marker loci. For example, the sequence of a nucleic acid comprising the marker can be stored in a computer. The desired marker locus sequence or its homolog can be identified using an appropriate nucleic acid search algorithm as provided by, for example, in programs as BLAST or any suitable sequence alignment tool.

The sequence of markers for QTLs according to the present invention preferably include 101 basepairs around the SNP identified with the marker. Specifically, a preferred sequence of the marker includes 50 base pairs on each of the 5′ and 3′ sides of the identified SNP, and the sequence of the 3′ plus 5′ segments is at least 95% identical to the sequence of the respective SEQ ID disclosed herein. Of course, the base at the SNP point will be one or the other of the two bases for the two alleles described herein for each of QTLs 1-7 and S1-S3.

Sequences that are at least 95% identical to the marker sequence can be easily detected in DNA recovered from seeds, plant parts or food samples by, for instance, comparison of sequences determined by NextGen sequencing methods, or by amplification-based assays using conditions for the annealing step that require at least 95% sequence identity for detection. Such methods are well known in the art, and include methods described herein, as will be understood by the skilled worker. Detection of a sequence at least 95% identical to the marker sequence will demonstrate the presence of the respective QTL in the seeds, plant parts and/or food products from which the DNA was obtained.

### Methods of Producing Sesame Plants

Methods are described herein for producing sesame plants or seeds comprising one or more introgressed shatter resistant capsule loci associated with a plurality of quantitative trait loci (“QTLs”) associated with shatter resistant capsules, wherein said plurality of QTLs comprises QTLs 1 to 7, and/or improved organoleptic properties associated with QTLs, wherein said QTLs comprise QTLs S1, S2, and S3. The method may comprise growing a sesame plant from the F1 seeds, crossing the F1 sesame plant with a sesame plant, and obtaining F2 seeds from the cross. The sesame plant of the present invention may have about 18% to about 25% protein content by weight, about 48% to about 56% fat content by weight, and/or an L value of greater than 60, as measured, for example, by Hunter Colorflex color meter in its seeds. Preferably, the sesame seeds may have about 20% to about 24% protein content by weight, about 50% to about 54% fat content by weight, and/or an L value of greater than 63, as measured, for example, by Hunter Colorflex color meter in its seeds.

A capsule of the sesame plant may comprise one or more introgressed shatter resistant capsule loci associated with a plurality of quantitative trait loci (“QTLs”) associated with shatter resistant capsules, wherein said plurality of QTLs comprises one or more QTLs 1 to 7, and/or QTLs associated with improved organoleptic properties, wherein said QTLs comprise one or more QTLs S1, S2, and S3.

A method for producing a sesame plant or seed, or a group of plants or seeds, is provided, whereby the plant, or group of plants, produce(s) a seed that may comprise one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, preferably at least two or all three of QTLs S1, S2, and S3. The seed may have about 18% to about 25% protein content by weight, about 48% to about 56% fat content by weight, and/or an L value of greater than 60, as measured, for example, by Hunter Colorflex color meter. Preferably, the seed may have about 20% to about 24% protein content by weight, about 50% to about 54% fat content by weight, and/or an L value of greater than 63, as measured, for example, by Hunter Colorflex color meter. The method comprises crossing two parent sesame plants or selfing a sesame plant and harvesting the resulting sesame seeds from the cross or selfing, wherein at least one parent is a sesame plant as described herein, or a derivative thereof. Seeds produced by the method are also provided herein, as are sesame plants produced by growing those seeds and sesame capsules harvested from those grown plants.

The method may further comprise the step of growing a F1 hybrid sesame plant obtained from seed obtained from said cross, crossing the F1 sesame plant to another sesame plant, e.g., to one of the parents used, and selecting progeny sesame plants comprising one or more introgressed shatter resistant capsule loci associated with a plurality of quantitative trait loci (“QTLs”) associated with shatter resistant capsules, wherein said plurality of QTLs comprises at least one, preferably at least 3 of QTLs 1 to 7, and/or QTLs associated with improved organoleptic properties, wherein said QTLs comprise one or more of QTLs S1, S2, and S3, and wherein the progeny sesame plants preferably produce sesame seeds having about 18% to about 25% protein content by weight, about 48% to about 56% fat content by weight, and/or an L value of greater than 60, as measured, for example, by Hunter Colorflex color meter. More preferably, the progeny sesame plants preferably produce sesame seeds having about 20% to about 24% protein content by weight, about 50% to about 54% fat content by weight, and/or an L value of greater than 63, as measured, for example, by Hunter Colorflex color meter.

The method may comprise the steps of:
(a) crossing a sesame plant producing sesame seeds comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and preferably the sesame seeds having about 18% to about 25% protein content by weight, about 48% to about 56% fat content by weight, and/or an L value of greater than 60, as measured, for example, by Hunter Colorflex color meter, and,
(b) obtaining the F1 seeds from said cross,
(c) selfing and/or crossing the plants obtained from the F1 seeds one or more times with one another or with other sesame plants, and
identifying and selecting progeny plants which produce seeds comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and the seeds preferably having about 18% to about 25% protein content by weight, about 48% to about 56% fat content by weight, and/or an L value of greater than 60, as measured, for example, by Hunter Colorflex color meter, and;
(d) phenotyping the seeds.

Optionally steps (c) and/or (d) can be repeated several times. Crossing in step (c) may also involve backcrossing. In step (d), plants producing seeds having about 18% to about 25% protein content by weight, about 48% to about 56% fat content by weight, and/or an L value of greater than 60, as measured, for example, by Hunter Colorflex color meter comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, may be selected. Thus, the one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, can also be used as selection criteria in addition to or as an alternative of shatter resistant capsule traits. The same applies to the methods described herein below, even if only shatter resistant traits are measured.

Phenotyping may comprise detecting one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, in the seeds (e.g., by phenotyping one or more populations of step c) above) and selecting rare recombinants or mutants which comprise one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3. The plants used under a) may be commercially available sesame plant cultivars or breeding lines. Phenotyping can be carried out on a plurality of single seeds independently, preferably grown under the same conditions next to suitable controls, or on a sample composed of (all or parts of) several seeds. When a single seed is used, preferably the mean value is calculated from a representative number of seeds. Phenotyping can be done one or more times. Phenotyping can be carried out at one or more steps of a breeding scheme.

Phenotyping may also comprise an analysis of the one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, in the sesame plants produced.

A method for making sesame plants comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, may comprise
(a) optionally, analyzing sesame seeds and/or capsules for one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3.
(b) crossing plants producing seeds comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, wherein the seeds may preferably have about 18% to about 25% protein content by weight, about 48% to about 56% fat content by weight, and/or an L value of greater than 60, as measured, for example, by Hunter Colorflex color meter, with sesame plants to produce F1 hybrids,
(c) selfing and/or (back)crossing F1 hybrid plants one or more times and
(d) selecting progeny plants comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, (at harvest and/or after storage) and preferably also for having shatter resistant capsules, and preferably also for producing seeds having about 18% to about 25% protein content by weight, about 48% to about 56% fat content by weight, and/or an L value of greater than 60, as measured, for example, by Hunter Colorflex color meter, and
(e) selecting a sesame plant producing seeds comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and preferably having about 18% to about 25% protein content by weight, about 48% to about 56% fat content by weight, and/or an L value of greater than 60, as measured, for example, by Hunter Colorflex color meter.

Step (d) may involve genetic analysis at harvest and/or after storage. In the initial cross, the sesame parent may be a sesame variety, cultivar or breeding line and the other plant may be a sesame variety, cultivar or breeding line. Preferably steps (c) and (d) are repeated several times, so that several cycles of phenotypic recurrent selection are carried out, leading to sesame plants of step (e).

A method of producing an inbred sesame plant comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and wherein the inbred sesame plant preferably produces seeds having about 18% to about 25% protein content by weight, about 48% to about 56% fat content by weight, and/or an L value of greater than 60, as measured, for example, by Hunter Colorflex color meter, may comprise:
(a) the creation of variable populations of Sesamum indicum comprising the steps of crossing a plant or plants producing seeds comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and the seeds preferably having about 18% to about 25% protein content by weight, about 48% to about 56% fat content by weight, and/or an L value of greater than 60, as measured, for example, by Hunter Colorflex color meter, with a plant of the species Sesamum indicum,
(b) harvesting the F1 seed from any of the plants used in the cross of (a) and growing F1 plants from the seed harvested,
(c) selfing the plants grown under b) or crossing these plants amongst one another, or crossing these plants with plants of Sesamum indicum,
(d) growing plants from the resulting seed harvested under normal plant growing conditions and,
(e) selecting plants producing seeds comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and the seeds preferably having about 18% to about 25% protein content by weight, about 48% to about 56% fat content by weight, and/or an L value of greater than 60, as measured, for example, by Hunter Colorflex color meter, followed by selfing the selected plants, and optionally
(f) repeating the steps (d) and/or (e) until the inbred lines are obtained which are homozygous and can be used as parents in the production of sesame plant hybrids comprising one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and wherein the sesame plant hybrids preferably produce seeds having about 18% to about 25% protein content by weight, about 48% to about 56% fat content by weight, and/or an L value of greater than 60, as measured, for example, by Hunter Colorflex color meter.

A method for producing a sesame seeds crop from sesame seeds or plants according to the invention and sesame seeds harvested therefrom is provided.

A method for producing a hybrid sesame seed plant comprising crossing the sesame plant comprising: one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, wherein the sesame plant preferably produces seeds having about 18% to about 25% protein content by weight, about 48% to about 56% fat content by weight, and/or an L value of greater than 60, as measured, for example, by Hunter Colorflex color meter, with another sesame plant, and obtaining a F1 sesame plant, wherein the F1 sesame plant one or more introgressed shatter resistant capsule loci associated with a plurality of quantitative trait loci (“QTLs”) associated with shatter resistant capsules, and wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and wherein the F1 sesame plant preferably produces seeds having about 18% to about 25% protein content by weight, about 48% to about 56% fat content by weight, and/or an L value of greater than 60, as measured, for example, by Hunter Colorflex color meter.

Sesame plants grown from the F1 sesame plant, wherein the F1 sesame plant comprises one or more introgressed shatter resistant capsule loci associated with a plurality of quantitative trait loci (“QTLs”) associated with shatter resistant capsules, and wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, and wherein the F1 sesame plant preferably produces seeds having about 18% to about 25% protein content by weight, about 48% to about 56% fat content by weight, and/or an L value of greater than 60, as measured, for example, by Hunter Colorflex color meter.

A method of producing sesame seeds may comprise planting seeds for a sesame plant comprising: one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improved organoleptic properties, wherein said QTLs comprise at least one, or at least two, or alternatively all three of QTLs S1, S2, and S3, wherein the seeds preferably have about 18% to about 25% protein content by weight, about 48% to about 56% fat content by weight, and/or an L value of greater than 60, as measured, for example, by Hunter Colorflex color meter, and harvesting the sesame seeds or capsules, growing, and harvesting the seeds. The harvesting may be done by machine.

Plant breeding methods are described in the art, for example, in U.S. Pat. Nos. 8,779,233; 6,670,524; 8,692,064; 9,000,258; 8,987,549; 8,637,729; 6,670,524; 6,455,758; 5,981,832; 5,492,547; 9,167,795; 8,656,692; 8,664,472; 8,993,835; 9,125,372; 9,144,220; 9,462,820; and U.S. Patent Application Publication Nos. 2015/0082476; 2011/0154528; 2014/0215657; 2017/0055481; 2015/0150155; and 2015/0101073.

Approaches for breeding the plants are described in the art. Selected, non-limiting approaches for breeding the plants are described below. A breeding program can be enhanced using marker assisted selection (MAS) of the progeny of any cross. It is further understood that any commercial and non-commercial cultivars can be utilized in a breeding program.

For highly inheritable traits, a choice of superior individual plants evaluated at a single location can be effective, whereas for traits with low heritability, selection can be based on mean values obtained from replicated evaluations of families of related plants. Popular selection methods commonly include, for example, but not limited to, pedigree selection, modified pedigree selection, mass selection, and recurrent selection. In a preferred embodiment, a backcross or recurrent breeding methods can be used.

The complexity of inheritance influences choice of the breeding method. Backcross breeding can be used to transfer one or a few favorable genes for a highly heritable trait into a desirable cultivar. This approach has been used extensively in breeding. Various recurrent selection techniques are used to improve quantitatively inherited traits controlled by numerous genes. The use of recurrent selection in self-pollinating crops depends on the ease of pollination, the frequency of successful hybrids from each pollination event, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new sesame cultivars requires the development and selection of sesame varieties, the crossing of these varieties and selection of superior hybrid crosses. The hybrid seed can be produced by manual crosses between selected male-fertile parents or by using male sterility systems, or by using differences between maternal and parental traits heritability in the seed as described in Israel Patent Application Publication IL239702 Hybrids are selected for certain single gene traits such as, for example, herbicide resistance which indicate that the seed is truly a hybrid. Additional data on parental lines, as well as the phenotype of the hybrid, may influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Pedigree breeding is used commonly for the improvement of self-pollinating crops. Two parents who possess favorable, complementary traits are crossed to produce a F1. A F2 population is produced by selfing one or several F1's. Selection of the best individuals in the best families is selected. Replicated testing of families can begin in the F4 generation to improve the effectiveness of selection for traits with low heritability. At an advanced stage of inbreeding (e.g., F6 and F7), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

Other suitable methods such as, for example, single-seed descent procedure and a multiple-seed procedure can also be used.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

In a multiple-seed procedure, breeders commonly harvest one or more capsules from each plant in a population and thresh them together to form a bulk. Part of the bulk is used to plant the next generation and part is put in reserve.

Other breeding methods are described in the art, for example, in Fehr, Principles of Cultivar Development Vol. 1, (1987).

The present invention further provides a sesame plant with improved organoleptic properties selected for by screening for sesame plant with improved organoleptic properties, the selection comprising interrogating genomic nucleic acids for the presence of a marker molecule that is genetically linked to an allele of a QTL associated with improved organoleptic properties in the sesame plant, where the allele of a QTL is also located on a linkage group associated with improved organoleptic properties.

### Sesame Plants and Parts Thereof

The sesame plants described herein are not naturally occurring sesame plants. Breeding efforts during the last seventy years have attempt to breed a mechanical harvestable sesame plant capsule have attempted using single gene mutations (ID, GS) and even a combination of few genes (ND and IND varieties). These efforts have failed, with the majority of the world's sesame (over 99%) being dehiscent (shattering) type. One reasons is that the breeding varieties that were developed using classical breeding methodology. Even with the changes in the sesame plants, there are still many agronomical problems such as low germination, plant lodging and low yield potential.

The present invention also provides a shatter resistant sesame plant selected for by screening for shatter resistance capsules plant, the selection comprising interrogating genomic nucleic acids for the presence of a marker molecule that is genetically linked to an allele of a QTL associated with shatter resistance capsules in the sesame plant, where the allele of a QTL is also located on a linkage group associated with shatter resistant sesame.

In an embodiment, a sesame plant or part thereof may comprise at least one quantitative trait loci (“QTLs”) associated with shatter resistant capsules, wherein the QTLs comprise QTLs 1 to 7 and the sesame plant may have about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter in its seeds. The sesame plant or part thereof may comprise at least three quantitative trait loci (“QTLs”) associated with shatter resistant capsules, wherein the QTLs comprise QTLs 1 to 7. The sesame plant or part thereof may comprise at least one, two, three, four, five, six, or seven of quantitative trait loci (“QTLs”) associated with shatter resistant capsules, wherein the QTLs comprise QTLs 1 to 7. The sesame plants comprising QTLs in their genome are not naturally occurring but have been created by a breeding program to create a new, non-naturally occurring sesame plant varieties.

The present invention also provides for a sesame plant with improved organoleptic properties selected for by screening for sesame plants with desirable organoleptic properties, the selection comprising interrogating genomic nucleic acids for the presence of a marker molecule that is genetically linked to an allele of a QTL associated with improved organoleptic properties in the sesame plant, where the allele of a QTL is also located on a linkage group associated with improved organoleptic properties in a sesame plant. A sesame plant or part thereof may comprise at least one quantitative trait loci (“QTLs”) associated with improved organoleptic properties, wherein the QTLs comprises one or more of QTLs S1, S2, and/or S3, and the sesame plant may have about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter, in its seeds. The sesame plant or part thereof may comprise all three quantitative trait loci (“QTLs”) associated with improved organoleptic properties, wherein the QTLs comprise QTLs S1, S2, and S3. The sesame plant or part thereof may comprise at least one or two of the quantitative trait loci (“QTLs”) associated with improved organoleptic properties, wherein the QTLs comprise QTLs S1, S2, and S3. The sesame plants comprising QTLs in their genome are not naturally occurring but have been created by a breeding program to create a new, non-naturally occurring sesame plant varieties.

This invention provides a sesame plant grown from a seed comprising: one or more introgressed shatter resistant capsule loci associated with a plurality of quantitative trait loci (“QTLs”) associated with shatter resistant capsules, wherein said plurality of QTLs comprises one or more of QTLs 1 to 7, and/or QTLs associated with improve organoleptic properties, wherein said plurality of QTLs comprise one or more of QTLs S1, S2, and S3, wherein the seed may have about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter.

The sesame plant may have shatter resistant capsules which are full or partial shatter resistant capsules. The sesame plant or part may be a hybrid.

Plants of the invention can be part of or generated from a breeding program. The choice of breeding method may depend on the mode of plant reproduction, the heritability of the trait(s) being improved, and the type of cultivar used commercially (e.g., F1 hybrid cultivar, pureline cultivar). A cultivar may refer to a variety of a plant that has been created or selected, and maintained through cultivation.

A sesame plant or a part thereof may comprise at least one introgressed shatter resistant capsule loci associated with a plurality of quantitative trait loci (“QTLs”) associated with shatter resistant capsules, wherein said plurality of QTLs comprises one or more of QTLs 1 to 7, and/or QTLs associated with improve organoleptic properties, wherein said plurality of QTLs comprises one or more of QTLs S1, S2, and S3, and wherein the sesame plant may have about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter, in its seeds. The sesame plant or part thereof may comprise at least three introgressed shatter resistant capsule loci associated with a plurality of quantitative trait loci (“QTLs”) associated with shatter resistant capsules, wherein the QTLs comprise QTLs 1 to 7, and/or QTLs associated with improve organoleptic properties, wherein said plurality of QTLs comprises one or more of QTLs S1, S2, and S3, and wherein the sesame plant may have about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter in its seeds.

In an embodiment, the present invention provides for a field comprising the sesame plant as described herein, wherein the sesame plant may have about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter, in its seeds and may comprise one or more introgressed shatter resistant capsule loci associated with a plurality of quantitative trait loci (“QTLs”) associated with shatter resistant capsules, wherein said plurality of QTLs comprises one or more of QTLs 1 to 7, and/or QTLs associated with improve organoleptic properties, wherein said plurality of QTLs comprises one or more of QTLs S1, S2, and S3.

The present invention also provides for parts of the plants of the present invention. Plant parts, without limitation, include seed, seed fragments (e.g., seeds that have been comminuted), endosperm, ovule and pollen. In a particularly preferred embodiment of the present invention, the plant part is a seed. In another embodiment of the present invention, the plant part is a seed fragment. The part may be a seed, an endosperm, an ovule, pollen, cell, cell culture, tissue culture, plant organ, protoplast, meristem, embryo, or a combination thereof.

This invention provides cells of the sesame plant comprising: one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improve organoleptic properties, wherein said plurality of QTLs comprises at least one, or at least two, or alternatively all three of QTLs S1, S2, and S3, and wherein the sesame plant has about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter in its seeds.

This invention provides seeds of the sesame plant comprising: one or more introgressed shatter resistant capsule loci associated with a plurality of quantitative trait loci (“QTLs”) associated with shatter resistant capsules, wherein said plurality of QTLs comprises at least one, preferably at least three of QTLs 1 to 7, and/or QTLs associated with improve organoleptic properties, wherein said plurality of QTLs comprises at least one, or at least two, or alternatively all three of QTLs S1, S2, and S3, wherein the seeds of the sesame plant have about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter.

Containers may comprise a plurality of sesame seeds and/or sesame capsules having the phenotypes described herein, as well as containers comprising a plurality of sesame seeds of the above plants or containers comprising a plurality of sesame plants or seedlings. Containers may be of any type, such as bags, cans, tins, trays, boxes, flats, and cargo totes. A container may contains at least about 1 pound, 5 pounds, 10 pounds or more of sesame seeds. The container may be in any location, e.g., a store (a grocery store), warehouse, market place, food processor, distributor.

In embodiments of this invention which include sesame seeds, all of the sesame seed may be from sesame plants of this invention. However, this invention also includes embodiments in which only part of the sesame seeds are from sesame plants of this invention. In such embodiments, at least 10% of the sesame seeds are from sesame plants of this invention. More preferably at least 15%, 20%, 25%, 30%, 35%, 40%, 45%, 50%, 55%, 60%, 65%, 70%, 75%, 80%, 85%, or even 90% are from sesame plants of this invention.

The sesame plant or a part thereof may comprise one or more of a plurality of markers associated with QTLs 1-7 and/or QTLs S1, S2, and S3, and the sesame plant may have about 18% to about 25%, preferably about 20% to about 24%, protein content by weight, about 48% to about 56%, preferably about 50% to about 54%, fat content by weight, and/or an L value of greater than 60, preferably greater than 63, as measured, for example, by Hunter Colorflex color meter in its seeds. Preferred sesame seeds and sesame plant parts according to this invention contain DNA having at least 3 of QTLs selected from QTL 1-7 and at least one QTL selected from S1, S2, and S3. More preferably, sesame seeds and/or sesame plant parts contain DNA having at least 3 of QTLs selected from QTL 1-7 and at least two or even all three of QTLs S1, S2, and S3. The preferences recited in this paragraph apply to the sesame plants, sesame seeds and sesame plant parts of all embodiments of this invention.

### Food Products

Sesame seeds and other plant parts described herein can be further processed to make a food product by any method known to one of skill in the art. This method may comprise heat treating, for example roasting, the plant parts, preferably sesame seeds. The method may further comprise comminuting, e.g., grinding, the seeds, including seeds following heat treated (roasting).

Food products comprising the sesame plant or part thereof may be made. Food products comprising a sesame seed paste or tahini may be made. For example, pet food products, ingredients (e.g., tahini), livestock feed, seed products, sauce, non-dairy milk product, spread, dip, jelly, cheese, cheese products, confection, candy, yogurt, carbonated beverages, non-carbonated beverages, baked good, pasta, dessert, cereal, snacks, salad, salad dressing, mix, flours, seasoning blends, toppings, bars, soups, soup bases, or combination thereof, may be made using the sesame plant or part thereof described herein. The plant part may include partially defatted seed.

The food product comprising sesame plant or part thereof may be animal feed, including but not limited to birdseed and livestock feed.

The food product may be a seed product including but not limited to a sprouted seed product, puffed sesame seed, roasted sesame seed, dehydrated sesame seed, raw sesame seed, or a combination thereof.

Spreads and dips including but not limited to hummus may be made using the sesame plant or part thereof described herein. A dip including but not limited to hummus or baba ganoush may be made using the sesame plant or part thereof described herein.

Food products including but not limited to bars, for example, nutritional bars, emergency food bar, nutraceutical bars, snack bars, breakfast bars, and meal replacement bars may be made using the sesame plant or part thereof described herein.

The sesame plant or part thereof described herein may be used to make confections and candy, for example halva and pasteli. Additionally, the sesame plant or part thereof described herein may be used to in making snacks, for example chips or snack sticks.

The sesame plant or part thereof described herein may be used to make baked goods including but not limited to bread, rolls, crackers, cookies, cakes, hamburger buns. For example, the sesame seeds described herein may be used as toppings for baked goods.

The sesame plant or part thereof described herein, preferably the seeds, may be used to make tahini. The tahini comprising the sesame seeds described herein may be used to make dips and spreads, including but not limited to hummus and baba ganoush.

The sesame plant or part thereof described herein may be used to in the making of cheese products including non-dairy cheese products. Additionally, the sesame plant or parts thereof described herein may be used in to make non-dairy milk products, for example, sesame seed milk. Also, non-carbonated beverage including but not limited to coffee and tea may comprise the sesame plant or part thereof described herein.

The sesame plant or part thereof described herein may be used to in the making of dessert including but not limited to ice cream, preferably ice cream comprising tahini made from the sesame seed plants or parts thereof described herein.

Further vitamins, supplements, thickeners, and binders may be made using the sesame plant or part thereof described herein or using a sesame seed paste or tahini containing sesame seeds produced by the sesame plant described herein.

Methods for making a food product comprising the sesame plant or part thereof described herein may comprise admixing ingredients and the sesame plant or part thereof described herein to produce a food product. The method may further comprise comminuting the sesame seeds. The method may further comprise roasting the sesame seeds. The method may further comprise comminuting the sesame seeds.

In an embodiment, the invention provides for a method of making a food product, such as tahini, comprising: selecting sesame seeds having about 18% to about 25% protein content by weight, about 48% to about 56% fat content by weight, and/or an L value of greater than 60, as measured, for example, by Hunter Colorflex color meter; and admixing the sesame seeds with ingredients to produce the food product. Preferably, the sesame seeds have about 20% to about 24% protein content by weight, about 50% to 54% fat content by weight, and/or an L value of greater than 63, as measured, for example, by Hunter Colorflex color meter.

In an embodiment, the invention provides for a method of making a raw material for a food product such as tahini, comprising: selecting sesame seeds having about 18% to about 25% protein content by weight, about 48% to about 56% fat content by weight, and/or an L value of greater than 60, as measured, for example, by Hunter Colorflex color meter. Preferably, the sesame seeds have about 20% to 24% protein content by weight, about 50% to 54% fat content by weight, and/or an L value of greater than 63, as measured, for example, by Hunter Colorflex color meter.

Several studies have indicated that significant genetic and environmental interactions may affect the composition of sesame seeds. The factor having the most significant impact on protein content is the accumulated growing degree days over the plant life cycle. Thus planting date and micro climate may be two important factors to growing the most flavorful sesame seeds for tahini manufacture.

Oil seed quality tests may be based on, or calibrated against, methods developed by internationally recognized standards writing agencies such as the American Oil Chemists' Society (AOCS). Oil contents of natural sesame seeds may be determined using industry standard Soxhet method, and protein contents may be determined using the Khejdal method. Results of wet chemistry analysis may be used to calibrate a near-infrared reflectance spectroscopy (NIRS) unit, and to establish a model of oil and protein content of intact natural sesame seeds. NIR spectroscopy is commonly used in the industry by seed breeders to rapidly analyze seed quality. The calibrated NIR unit may be, for example, Perten Model 7250.

“To date there has not been much trading based on seed contents, but some markets are becoming conscious of the components. Some of the variations in the seed include: protein from 19% to 30% and oil from 34.4% to 59.8% (Ashri 1998).” D. Ray Langham and Terry Wiemers, Progress in Mechanizing Sesame in the US Through Breeding, Trends in New Crops and New Uses. 2002. J. Janick and A. Whipkey (eds.). ASHS Press, Alexandria, Va. An aspect of the sesame plant or part thereof described herein is that the protein content and the oil content of the sesame seeds may be inversely correlated. As the protein levels increase, the crude oil content decreases. Protein and oil components, along with natural sugars are key precursors to volatile flavor compounds formed during roasting and milling of sesame. Using non-destructive NIR and color measurement equipment, seed selection for tahini manufacture may rapidly be performed at the field level, at receiving by the grain processor, and or during the seed cleaning process using benchtop, handheld or in-line equipment known to the industry.

In one embodiment, a method of making tahini may comprise roasting and comminuting the sesame seed described herein. The sesame seeds may be roasted before comminuting. The sesame seeds may be comminuted and then roasted. The method for making a food product comprising the sesame plant or part thereof, preferably the seeds, may comprise cleaning said sesame seeds, washing, drying, dehulling, roasting, and comminuting said sesame seeds.

In an embodiment, the invention provides for a composition that comprises or consists of tahini, wherein the tahini includes sesame seeds comprising introgressed organoleptic property loci associated with a plurality of quantitative trait loci (QTLs) associated with organoleptic properties, wherein said plurality of QTLs comprise S1, S2, S3, or a combination thereof, comprising introgressed shatter resistant capsule loci associated with a plurality of quantitative trait loci (QTLs) associated with shatter resistant capsules, wherein said plurality of QTLs associated with shatter resistant capsules comprise at least one of QTL 1, 2, 3, 4, 5, 6, 7, or a combination thereof, and having a protein content of about 18% to about 25% by weight, a fat content of about 48% to about 56% by weight, and/or an L value of greater than 60 as measured, for example, by Hunter Colorflex color meter. Preferably, the sesame seeds have about 20% to about 24% protein content by weight, about 50% to about 54% fat content by weight, and/or an L value of greater than 63, as measured, for example, by Hunter Colorflex color meter.

In embodiments of this invention which include sesame seeds and/or sesame plant parts, all of the sesame seed and/or sesame plant parts may be from sesame plants of this invention. However, this invention also includes embodiments in which only part of the sesame seed and/or sesame plant parts are from sesame plants of this invention. In such embodiments, at least 10% of the sesame seed and/or sesame plant parts are from sesame plants of this invention. More preferably at least 15%, 20%, 25%, 30%, 35%, 40%, 45%, 50%, 55%, 60%, 65%, 70%, 75%, 80%, 85%, or even 90% are from sesame plants of this invention. It has been observed that improved organoleptic characteristics associated with seeds and or plant parts obtained from sesame plants having one or more of the preferred alleles may be detected when as little as 10% of the sesame-derived material in a food product is from such plants, and a greater percentage of the sesame-derived material coming from such plants may result in greater improvement.

The relative percent of sesame-derived material in a product that comes from a sesame plant according to the present invention may be determined by any method known to the skilled worker for distinguishing plant material according to this invention from other sesame material. Such methods may include quantitative measurement of the DNA sequences of QTLs according to this invention compared to an unrelated DNA sequence that is highly conserved in the sesame genome. Such sequences are disclosed in, e.g., Wang et al. 2014) Genome Biology 15(2): R39.

Further embodiments of the present invention will now be described with reference to the following examples. The examples contained herein are offered by way of illustration and not by any way of limitation.

### EXAMPLES

### Example 1

### QTL for Shatter Resistant Sesame

This innovation presents a methodology of breeding sesame lines bearing shatter resistant capsules. Sesame plants grown worldwide are harvested manually. The first and foremost obstacle to complete mechanization for this important crop is the dehiscence nature of its capsules. This innovative is based on the collection of worldwide sesame lines, the creation of F2 linkage populations, massive phenotyping and genotyping of thousands of sesame lines, prediction of QTL's affecting the shattering resistance trait, and the establishing of unique marker combinations (a “marker cassette”) for shattering resistant sesame lines never found before in commercial or natural lines.

The breeding methodology is based on discovery of the Target Product Genomic Code (TPGC). The Target Product (TP) is define in advance based on market requirements; it includes a set of desired attributes (traits) that are available in natural genetic variations. The Genomic Code (GC) is a set of genomic regions that affect the Target Products' traits. Proprietary algorithms take the GC, which is composed of a quantitative trait locus (QTL) database linked to the TP, and define the Target Product Genomic Code (TPGC). The algorithms calculate multiple genomic interactions, including effects of heterosis and epistasis, and maximize the genomic potential of specific plants for the development of new varieties. The breeding program discovers the TPGC, then by crossing and selfing progresses until a product is achieved which contains the specific GC discovered to be linked to the TP. A typical breeding project includes the following breeding and technical cycles:

Trait Discovery—where a broad spectrum of varieties from different geographies and worldwide sources are grown and phenotyped in order to discover new traits that can potentially be combined to create the new product.

Trait Blend—a crossing cycle based on phenotypic assumption, where the different traits are mixed and combined. The initial trait cycle is followed by an additional cycle to create a F2 population, which will provide the basis for algorithmic analysis that will lead to the TPGC construction.

TPGC Discovery—the most important phase where every single plant is phenotyped and genotyped to produce a linkage map, discover the QTLs and discover the TPGC using proprietary technology.

Line Validation 1.1—the first year of validating line version 1. These lines are based on millions of in silico selections and are defined as the project's pioneer varieties.

Line Validation 1.2—the second year of validating line version 1.

Pre-commercial 1.3—the third year and final validation of line version 1.

Trait TPGC Blend—in this the phase accurate crossing based on the proprietary algorithm was performed, calculating the most efficient way to reach the best TPGC. The crossing is performed after in silico selection of millions of combinations. The trait TPGC blend phase is followed by an additional cycle to produce a F2 population for a second GC discovery. It is important to note that this phase is based on the proprietary algorithm, unlike the Trait Blend phase that is based on phenotype assumptions. Defining the TP for sesame include identifying the shatter resistant trait to enable harvesting mechanically. To identify the shatter resistant capsules traits, a set of phenotype traits were developed to correlate with measured seed retention and capsule structure. The unique combination between the capsule structure and seed retention enable it to be harvested mechanically but still enabling the seed to release easily by the thresher in the combine. For the unique combination, identifying a plurality of quantitative trait loci (“QTLs”) associated with it (GC) completes the TPGC for breeding sesame for mechanical harvesting.

The trait discovery is based on germplasm which included five hundred different sesame lines that were obtained from the U.S. National Plant Germplasm System (NPGC) and courtesy of Prof Amram Ashri's sesame germplasm collection (Ashri, 1998). Screening for trait discovery was based on allocating traits related to capsule structure and capsule retention of the seeds.

150 different lines were produced for trait blend—crosses, executed based on the potential for enrichment of genomic diversity as creating a new complex of traits for the shatter resistant capsules as the initial step for a TP directed breeding program for shattering resistant sesame lines. The resulted F1 hybrids were later self-crossed to create F2 linkage populations that showed phenotypic segregation and a combination of QTLs (1-7) not found in nature.

The F2 population was then planted in 6 different environments for discovering the TPGC, including shattering resistant capsules traits. After screening 15000 individuals, a set of 3000 representatives was selected. The selected F2 individuals were massively phenotyped for three shatter resistant capsule (SRC) components:

SRC1: Evaluating the rate of the seed retention by shaking the plant and counting the amount seeds that are falling down to the ground.

SRC2: Evaluating the rate of the seed retention after the capsules are turned upside down, by counting the amount of the seeds that remain inside the capsules.

SRC3: Measuring the ratio between the total length of the capsule and the length of the zone in which the capsule tips are open, by measuring each of the lengths using a ruler.

All the shatter resistant capsule trait's components were summarized into one representative trait which was named the shatter resistant capsule trait. The selected 3000 individuals were genotyped under examination of a panel with 400 markers, based on single nucleotide polymorphism (SNP). This 400 marker panel was directly designed based on parental lines RNA-sequences of each linkage F2 population. The panel was designed to maximize the chance to have the largest number of common segregate SNP's in order to create highly similar linkage maps for all observed populations.

### Mapping Population

The computation of linkage maps were executed on each linkage F2 population based on genotyping results. Linkage maps were computed with MultiPoint, an interactive package for ordering multilocus genetic maps, and verification of maps based on resampling techniques.

### QTL Discovery

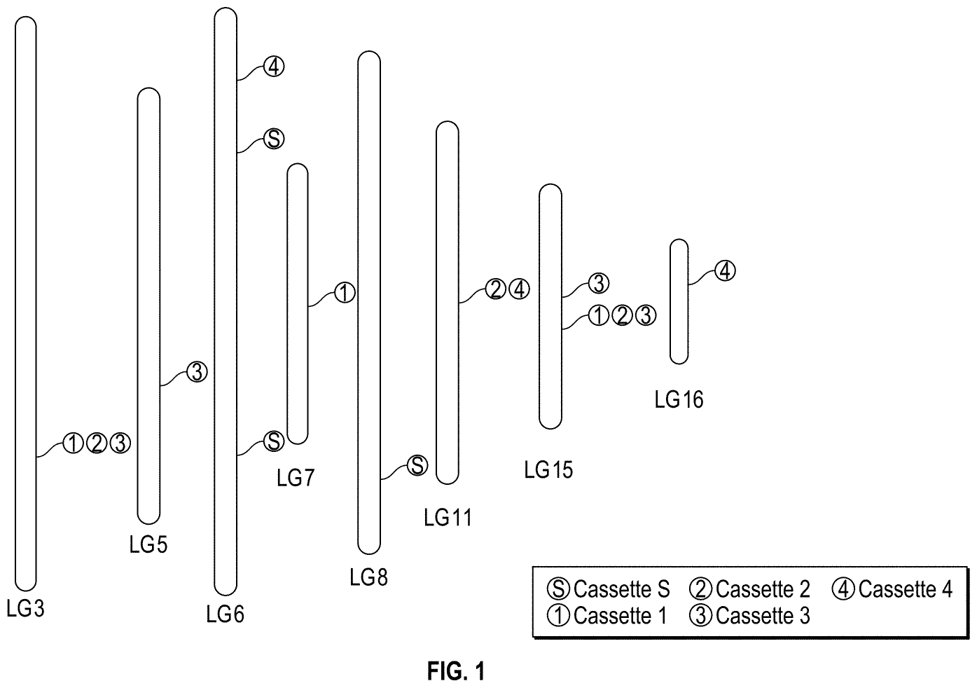

QTL discovery related to shattering resistance was executed with MultiQTL package. The program produced linkage maps that were merged by Multipoint and the F2 population phenotype data. MultiQTL use multiple interval mapping (MIM). MultiQTL significance is computed with permutation, bootstrap tools and FDR for total analysis. The linkage maps of all eight F2 populations and the information of the three shatter resistant capsule traits over all genotyped plants belonging to those population were analyzed. The prediction of QTL was in a “one trait-to-one marker” model, meaning that for all markers that constructed the linkage maps, each trait was tested independently against each one of the markers. The results point to 8 markers from 7 different linkage group that are representing QTL's related to shattering resistance as described in Table 1. Each population presented a different marker cassette related to shattering resistant but still some populations shared a subset of common markers with other populations. The verities of marker cassettes were summarized as described in FIG. 1.

### Significance and Co-Occurrences of Shattering Resistant Capsules Markers

The QTL analysis provided the set of markers that represent QTL related to shattering resistant capsules in sesame for each linkage F2 population separately. In order to strengthen the significance of each marker, an in-house algorithm was developed to observe genotype-phase of each marker related to QTL/trait in all linkage F2 populations in different environments. The occurrence of shattering resistance capsule markers in two or more linkage F2 populations (repetitive markers) strengthen its significance as representative for shattering resistant capsules QTL. In addition, the co-occurrence of non-repetitive and repetitive markers related to shattering resistance capsules in a given population was observed for the design of “marker cassettes” that provide the genetic signature for shattering resistant capsules in sesame lines.

### In-Silico Self- and Cross-Self Based Breeding Program

Based on the QTL prediction, which provide the effect of each phase of a given marker for each of the three shatter resistant capsule traits, three different algorithms for the simulation and prediction of the genotypic state of self, cross-self and hybrid plant was developed in-house for processing the TPGC blend. The TPGC blend combines QTL's from different populations together into a single plant to increase similarity of the discovered TPGC to an exciting product, which contains a unique cassette of QTL's for shatter resistant capsule which never exist before. The algorithms design in silico millions of selfing combination from F2 to F8, millions of new combination of F1 and then selfing to F8 and millions of F1 hybrids to create hybrid variety. This was done in order to measure the potential for each of the 3000 plants to acquire the shatter resistant capsules in the right combination at the right phase. After running the analysis among ˜3000 plants, 200 higher score plants were chosen for the selfing, cross selfing and hybrid programs.

### Validation of Shatter Resistant Capsules Lines

After the determination of which plants have the highest potential to acquire shattering resistant capsules based on genetic code, it is important to preserve this potential in next generations. In order to follow the genetic code of the shattering resistant capsules “marker cassettes”, the offsprings of each chosen lines (the next generation) were genotyped based on the shattering resistant capsules “marker cassettes”. Only offsprings that present the previous generation “marker cassette” for shattering resistant capsules were selected and forwarded to the next generation. This procedure ensures the maintenance of the shattering resistant capsules trait and “marker cassette” for each shattering resistant line. This invention presents a methodology for the design of 4 marker-cassettes that point, with one marker cassette or more, on shattering resistant capsules sesame lines.

TABLE 1
Marker cassettes and QTL

| | | | | Reference(1) | alternative | | | | | P-value |
|---|---|---|---|---|---|---|---|---|---|---|
| Marker name | LG | Position | SRC(2) | allele | allele | cassette1 | cassette2 | cassette3 | cassette4 | (3) |
| LG3_19205572 | 3 | 19205572 | SRC3 | C | T | CC/CT | CC/CT | CC/CT | — | 0.05 |
| LG5_12832234 | 5 | 12832234 | SRC3 | C | T | — | — | CC/CT | — | 0.025 |
| LG6_2739268 | 6 | 2739268 | SRC3 | T | C | — | — | — | CC/CT | 0.045 |
| LG7 5141423 | 7 | 5141423 | SRC1, SRC3 | C | G | CC/CG | — | — | — | 0.0075 |
| LG11_8864255 | 11 | 8864255 | SRC3 | C | G | — | CC/CG | — | CC/CG | 0.003 |
| LG15_4900868 | 15 | 4900868 | SRC1, SRC2, | G | A | — | — | AA/AG | — | 0.0005 |
| | | | SRC3 | | | | | | | |
| LG15_5315334 | 15 | 5315334 | SRC1, SRC2, | T | C | CC/CT | CC/CT | CC/CT | — | 0.0005 |
| | | | SRC3 | | | | | | | |
| LG16_1563304 | 16 | 1563304 | SRC3 | A | G | — | — | — | GG/AG | 0.038 |

(1)Reference allele based on Sesamum indicum reference Genome V1.0 (Wang L, Yu S, Tong C, et al. Genome sequencing of the high oil crop sesame provides insight into oil biosynthesis. Genome biology, 2014, 15(2): R39).
(2)The SRC trait that is effected by a given marker.
(3)The p-value is the significance level of single- QTL analysis commuted by MultiQTL program.

TABLE 2
Heterozygous Allele Effect

| | | | Alternative Allele | | Heterozygous | | |
|---|---|---|---|---|---|---|---|
| | Reference Allele Effect | | Effect | | Allele Effect | | |
| Marker name | Effect | STD | Effect | STD | Effect | STD | p-value |
| LG3_19205572 | 141 | 25 | 94 | 27 | 132 | 24 | 0.05 |
| LG5_12832234 | 18.4 | 1.4 | 12.5 | 1.25 | 14.2 | 1.8 | 0.025 |
| LG6_2739268 | 13.8 | 1.2 | 17.6 | 1.8 | 13.3 | 2.45 | 0.045 |
| LG7_5141423 | 17.4 | 1.1 | 14.2 | 1 | 12.2 | 1.5 | 0.0075 |
| LG11_8864255 | 25.8 | 1.6 | 21.4 | 0.8 | 22.8 | 0.9 | 0.003 |
| LG15_4900868 | 14.5 | 0.6 | 28 | 0.6 | 24.7 | 0.8 | 0.0005 |
| LG15_5315334 | 13.6 | 0.4 | 22.1 | 0.55 | 20.5 | 0.65 | 0.0005 |
| LG16_1563304 | 23.4 | 2 | 32 | 2.3 | 23.1 | 2.9 | 0.038 |

### Example 2

### Breeding of Improved Sesame Seed Plants

A Breeding Program using the method described in Example 1 was carried out using sesame plants having one or more of QLTs 1-7. Sesame plants comprising QTL1-7 were selected because of their shatter-resistant seed pod and adaptability to agronomic practices for both dryland and irrigated production methods. The plants were crossed and grown as described in Example 1 and screened for the desired color, fat and protein content and organoleptic characteristics, such as the suitability of the lines to be converted into tahini.

Preferably, the sesame plant has a protein content of from about 24.5% to 28.4% and a fat content of from about 44.5% to 50.3%. Alternatively, the sesame seed has a protein composition of about 23%+/−2% and a fat composition of about 50%+/−2%.

The seeds may be off-white or white in color. The technique to measure seed color uses the “Lab” color space—a color space defined by the International Commission on Illumination (CIE) in 1976. It expresses color as three numerical values, L* for the lightness and a* and b* for the green-red and blue-yellow color components. CIELAB was designed to be perceptually uniform with respect to human color vision, meaning that the same amount of numerical change in these values corresponds to about the same amount of visually perceived change. The lightness value, L*, represents the darkest black at L*=0, and the brightest white at L*=100. The color channels, a* and b*, represent true neutral gray values at a*=0 and b*=0. The a* axis represents the green-red component, with green in the negative direction and red in the positive direction. The b* axis represents the blue-yellow component, with blue in the negative direction and yellow in the positive direction. The seeds of the sesame plants described herein may have a seed with seed color values ranges of more than 71 color L and a range of 4 to 5.5 of color B and 10 to 15 color B making the seed whitish in appearance.

For organoleptic analysis, the seeds of each line were toasted, ground to a paste, and mixed with olive oil to make tahini, which was evaluated by a trained taste panel for comparison to control tahini made from commercial sesame seeds grown in Ethiopia in the Humera region.

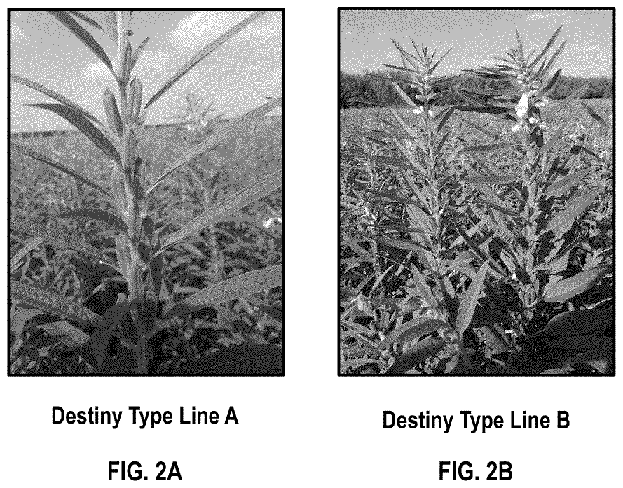

The lines that meet these characteristics were found to comprise the presence of one or more of QTL S1, S2 and S3. These sesame seed plants were selected. See, e.g., FIG. 2A-2B. Plants which meet the preferred protein, fat, and color criteria and contain one or more, particularly two of more, or even all three of QTL S1, S2, and S3 are plants of this invention.

### Example 3

### Organoleptic Characterization of Improved Sesame Seed Plants

Seven sesame seed plant lines were selected in Example 2 were grown in two geographically distinct semi-arid areas with similar agronomic characteristics. Two lines, Destiny Type Line A and Destiny Type Line B were selected for further breeding and characterization.

The selected lines were all shatter resistant (e.g., the sesame plants can be harvested by machine) and have yields that are superior to Ethiopia Humera lines. Sesame seed yields can vary widely depending on agricultural practices. In Africa, sesame yields have an average yield of 267 to 500 lbs/acre. Berhane Girmay, A. University of Aksum/Hawass University. Sesame production, Challenges and opportunities in Ethiopia, December 2015. Two sesame seed lines, Destiny Type Line A and Destiny Type Line B showed a yield ranging from 600 to 1,800 lbs per acre.

The selected sesame plant lines that exhibit a branching type and/or a seed count per pod count that is higher than commercially available lines. To develop a reference, a wide sample of germplasm from commercially available seeds was obtained and found to exhibit a large phenotypic variation that was classified as at least 10 different varieties. Seeds from the most common phenotype were collected as a “Control”. Control varieties flowering under long day growing conditions. Commercially available sesame seed varieties exhibit an initial flowering range between 15 to 25 cm above ground. Control plants have an average of 30 capsules on its main branch, they have an average of 12 lateral branches that each carry 15 capsules which sum up to 210 capsules. Selected sesame seed lines have several types of phenotypic expressions that can range between 180 to 240 capsules in its main branch, an average of 5 lateral branches and a range between 400 and 600 total capsules per plant. Further, the sesame seed lines express initial flowering at 80 cm above ground as compared to other sesame seed varieties that range between 15 to 25 cm above ground.

The portfolio of seeds grown were assessed for sensory characteristics using a trained panel using a modified spectrum descriptive analysis methodology scale for sesame seed using literature readily available and described in Sensory Evaluation Techniques (4^{th} Edition) Meilgaard, Carr, & Civille CRC Press (2007).

Sensory results are then analyzed by assigning numeric scale values to positive and negative sensory characteristics and then weighting the characteristics in order of importance to generate a specific score. Seeds produced by plants that do not meet minimum sensory characteristics score of high sweetness, low bitterness and no off-tastes are then rejected, and remaining seed portfolio is then evaluated a second time using a different method.

The remaining portfolio of seeds is processed by manufacturing a small batch of tahini using benchtop or small factory methods as described in tahini manufacturing protocols found in the arts and then evaluated by trained panel using a more detailed spectrum descriptive analysis methodology.

TABLE 3
Protein and Fat Analysis of Sesame Seeds

| Destiny Type | |
|---|---|
| Line A | Line B |

| | Protein (%) | Fat (%) | Protein (%) | Fat (%) |
|---|---|---|---|---|
| Farm A | 24.5 | 50.3 | 25.2 | 49.0 |
| Farm B | 28.4 | 45.0 | 24.6 | 44.5 |

The seeds were whitish in color. Seed color is evaluated for breeding of sesame varieties because it affects the quality and appeal of processed seeds.

The color description is based on the use the color spectrum analysis graph that uses color L, color A and Color B outlined as follows. The “Lab” color space is a color space defined by the International Commission on Illumination (CIE) in 1976. It expresses color as three numerical values, L* for the lightness and a* and b* for the green-red and blue-yellow color components. CIELAB was designed to be perceptually uniform with respect to human color vision, meaning that the same amount of numerical change in these values corresponds to about the same amount of visually perceived change. The lightness value,L*, represents the darkest black at L*=0, and the brightest white at L*=100. The color channels, a* and b*, represent true neutral gray values at a*=0 and b*=0. The a* axis represents the green-red component, with green in the negative direction and red in the positive direction. The b* axis represents the blue-yellow component, with blue in the negative direction and yellow in the positive direction.

Chroma meters such as the Konica Minolta's BC-10 are standard tools for accurate color determination. Designed for direct contact measurements, the BC-10 is not affected by lighting conditions and eliminates inconsistencies such as human eye sensitivities. For standardized, comparable color measurements of seeds, chroma meters measure in a device independent color space made up of three channels: L*, which ranges from 0 to 100 and represents the lightness of the color; a*, negative or positive values of which represent green or magenta, respectively; and b*, representing blue (negative) or yellow (positive). These channels can then be used individually to quantify specific color attributes, which may be linked to biological factors. To measure seed color using the BC-10 handheld chroma meter 1. Switch on the power. 2. Perform white tile calibration. 3. Place on product and press button. 4. Measurement results are displayed immediately. Three to 5 readings are typically taken, and the average reading reported

Utilizing the LAB color space methodology as defined by CIE, the inventors determined that seed was whitish in color. The inventors took these measurements in our laboratory using a handheld colorimeter following established protocols to measure seed color.

TABLE 4
Color Analysis of Sesame Seeds

| Sesame Seed Plant | Color L | Color A | Color B |
|---|---|---|---|
| Destiny Type Line A | 71.96 | 4.01 | 10.93 |
| Control | 72.79 | 5.29 | 14.18 |
| Destiny Type Line B | 71.65 | 5.54 | 13.68 |

Seed that have QTL 1-7 and have shown general agronomic traits of yield potential are assessed for sensory characteristics using a modified spectrum descriptive analysis methodology scale developed specifically for sesame seed using methods described in literature readily available and described in a book called: “Sensory Evaluation Techniques, fourth edition: Meilgaard M., Civille G., Carr T.” Seeds that do not meet minimum sensory characteristics of, low bitterness and no off-tastes are then rejected and remaining seed portfolio is then evaluated a second time.

To develop a reference, a wide sample of germplasm from commercially available seeds was obtained and found to exhibit a large phenotypic variation that was classified as at least 10 different varieties. Seeds from the most common phenotype were collected as a “Control”. The color of the control represents the aggregate of seeds selected for a preferred color.

The remaining portfolio of seeds is processed by manufacturing small batches of comminuted tahini paste (tahini) using laboratory tools or small factory methods known in the art and then evaluated by using a trained panel and a more detailed spectrum descriptive analysis methodology. Tahini that meet a minimum standard of sweet roasted flavor, nutty flavor, low bitterness and no off-tastes are then selected.

### Example 4

Using the Sesame Screener for protein, oil contents and color, 25 varieties from the 2018 Crop Year having the target composition and color were chosen for detailed flavor analysis by trained sensory panelist. All seeds were cleaned, dehulled, dried then roasted under repeatable laboratory conditions prior to sensory evaluation. The roast seeds were then milled into tahini and evaluated against 4 key sensory attributes using a 10 point hedonic scale. Attributes included basic tastes of Sweet and Bitter, and Sweet Roasted and Nutty aromatics. Panelist also gave each variety an overall Pass or Fail rating for acceptability for tahini manufacture. Roast seeds were also evaluated using electronic nose technology to identify key aroma compounds and to provide an aroma finger print of each sesame variety. Multivariate data analysis using JMP statistical software was then used to establish the importance to protein, oil, and seed color on sensory Pass rating and on Sweet Roast and Nutty Aromatics. Roughly 70% of the chosen sesame varieties were given a passing rating by the panelists showing the value of the screener. E-nose results also identified key compounds developed during roasting that contributed to desired Sweet Roasted and Nutty aromatics. Optimizing seed processing and roasting conditions based on flavor chemistry and roast aroma will only further enhance flavor of seeds having the target composition and color. These results bolster the use of rapid quality measures such as NIR composition, E-nose aroma analysis and color measurements to aid selection of sesame for tahini production.

Oil seed quality tests were based on, or calibrated against, methods developed by internationally recognized standards writing agencies such as the American Oil Chemists' Society (AOCS). Oil contents of natural sesame seeds were determined using industry standard Soxhet method, and protein contents were determined using the Khejdal method. Results of wet chemistry analysis was used to calibrate a near-infrared reflectance spectroscopy (NIRS) unit, and to establish a model of oil and protein content of intact natural sesame seeds. NIRS predictions compared to reference results agreed excellently, providing a rapid and efficient method to accurately determine oil and protein content of sesame varieties. NIR spectroscopy is commonly used in the industry by seed breeders to rapidly analyze seed quality. The calibrated NIR unit used in this work was a Perten Model 7250.

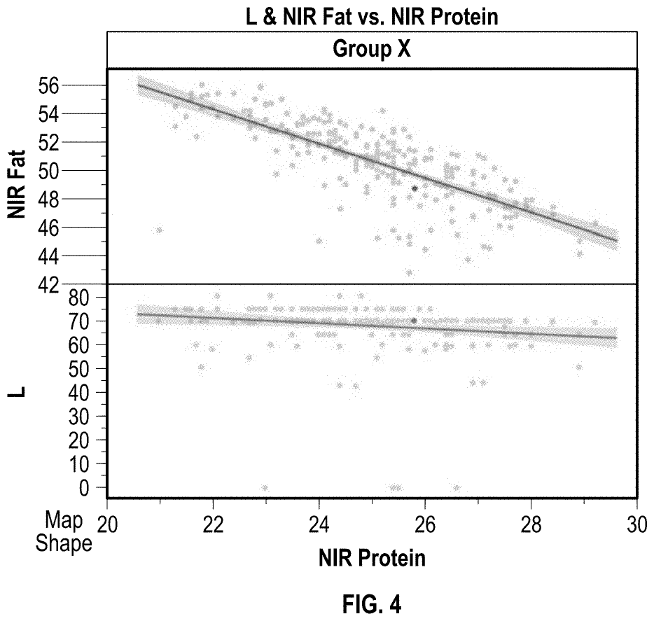

The relationship between seed color as indicated by L value and fat content of the sesame seeds compared to protein content of the sesame seeds is shown in FIG. 4.

For the 2018 Crop Year 93 new varieties were evaluated. 85 varieties had protein less than 24% protein. Only 26 varieties with protein less than 24% were whitish with an L value of greater than 69 as determined using Minolta BC10, which is equivalent to an L value of 60 using a Hunter color meter (see Table 5).

TABLE 5
Protein and Sensory Analysis of Sesame Seeds

| 2018 Crop Year | | Tahini Sensory P/F, Bitter, Sweet | | |
|---|---|---|---|---|
| Data | | Roast & Nutty | | |
| | Protein | Tahini Sensory | | Sweet |
| Variety | (%) | Pass/Fail | Bitter | Roasted + Nutty |
| C409 | 20.95 | Pass | 1.1 | 5.2 |
| C732 | 22.83 | Pass | 1.3 | 4.4 |
| C660 | 22.96 | Pass | 0.9 | 4.8 |
| C184 | 21.08 | Pass | 1.2 | 5.6 |
| C420 | 20.71 | Pass | 1.2 | 4.4 |
| C484 | 21.96 | Pass | 1.2 | 4.7 |
| C956 | 20.97 | Pass | 1.1 | 4.0 |
| C848 | 22.68 | Pass | 1.5 | 3.9 |
| C880 | 22.06 | Pass | 1.5 | 4.5 |
| C455 | 20.99 | Pass | 0.8 | 5.0 |
| C275 | 19.73 | Pass | 1.7 | 4.3 |
| C412 | 21.98 | Pass | 1.2 | 4.6 |
| C547 | 22.72 | Pass | 1.4 | 3.9 |
| C730 | 21.35 | Pass | 1.3 | 4.5 |
| C871 | 21.26 | Pass | 1.5 | 4.2 |
| C890 | 22.61 | Pass | 1.1 | 4.4 |
| C936 | 21.33 | Pass | 1.1 | 5.3 |
| C348 | 20.16 | Fail | 1.3 | 3.3 |
| C458 | 22.32 | Fail | 2.2 | 3.7 |
| C496 | 22.15 | Fail | 1.8 | 4.1 |
| C285 | 23.24 | Fail | 2.3 | 3.5 |
| C511 | 22.65 | Fail | 1.9 | 4.1 |
| C560 | 22.87 | Fail | | |
| C683 | 23.96 | Fail | | |
| C986 | 22.73 | Fail | | |

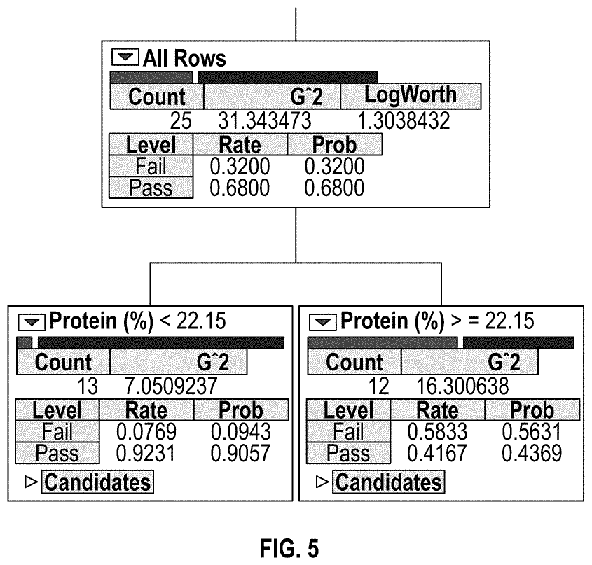

FIG. 5 shows the JMP partition decision tree for 25 sesame varieties having protein contents averaging 20 to 24% in 2018 crop year. Roast Seeds were milled into tahini for sensory evaluations.

Although the invention has been described in some detail by way of illustration and example for purposes of clarity of understanding, it should be understood that certain changes and modifications may be practiced within the scope of the present disclosure. Modifications of the above-described modes for carrying out the invention that would be understood in view of the foregoing disclosure or made apparent with routine practice or implementation of the invention to persons of skill in food chemistry, food processing, mechanical engineering, and/or related fields are intended to be within the scope of the present disclosure.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

We claim:

1. A sesame plant or a part thereof, comprising introgressed organoleptic property loci associated with a plurality of quantitative trait loci (QTLs) associated with organoleptic properties, wherein said plurality of QTLs comprise S1, S2, S3, or a combination thereof, having a protein content of about 18% to about 25% by weight, a fat content of about 48% to about 56% by weight, and/or an L value of greater than 60 as measured by Hunter Colorflex color meter in its seeds, and comprising one or more introgressed shatter resistant capsule loci that are associated with a plurality of quantitative trait loci (QTLs); wherein the plurality of QTLs comprises QTL 1, QTL 2, QTL 3, QTL 4, QTL 5, QTL 6, and QTL 7; wherein the QTL 1 comprises the nucleic acid marker of SEQ ID NO: 1 or 9, the QTL 2 comprises the nucleic acid marker SEQ ID NO: 2 or 10, the QTL 3 comprises the nucleic acid marker of SEQ ID NO: 3 or 11, the QTL 4 comprises SEQ ID NO: 4 or 12, the QTL 5 comprises the nucleic acid marker of SEQ ID NO: 5 or 13, the QTL 6 comprises the nucleic acid markers of SEQ ID NO: 6 or 14 and SEQ ID NO: 7 or 15, and the QTL 7 comprises the nucleic acid marker of SEQ ID NO: 8 or 16; wherein said nucleic acid markers are arranged in a marker cassette; and wherein said nucleic acid markers are associated with shatter resistance capsules.

2. The sesame plant or a part thereof according to claim 1, wherein said shatter resistant capsules comprise a fully or partly shatter resistant capsules.

3. The sesame plant or a part thereof according to claim 1, further comprising alleles of said nucleic acid markers associated with said plurality of QTLs.

4. The sesame plant or a part thereof according to claim 3, wherein the alleles of one or more of said markers are homozygous or heterozygous.

5. The sesame plant or a part thereof according to claim 3, wherein said marker cassette comprises marker cassette 2 comprising the nucleic acid marker SEQ ID NO: 9, 13, 15 or a combination thereof, and wherein the alleles for said nucleic acid markers are homozygous or heterozygous.

6. The sesame plant or a part thereof according to claim 3, wherein said marker cassette comprises marker cassette 4 comprising the nucleic acid marker SEQ ID NO: 5, 11, 16 or a combination thereof, and wherein the alleles for said nucleic acid marker are homozygous or heterozygous.

7. The sesame plant or part thereof according to claim 1, wherein said part is a seed, an endosperm, an ovule, pollen, cell, cell culture, tissue culture, plant organ, protoplast, meristem, embryo, or a combination thereof.

8. The sesame plant or part thereof according to claim 1, wherein said plant is a hybrid.

9. The sesame plant or a part thereof according to claim 1, wherein said marker cassette comprises cassette 2, 4, or a combination thereof, wherein said cassette 2 comprises the nucleic acid marker SEQ ID NO: 9, 13, 15 or a combination thereof, wherein said cassette 4 comprises the nucleic acid marker SEQ ID NO: 5, 11, 16, or a combination thereof.

10. A method of producing shatter resistant sesame plants, said method comprising: a) providing a sesame plant comprising introgressed organoleptic property loci associated with a plurality of quantitative trait loci (QTLs) associated with organoleptic properties, wherein said plurality of QTLs comprise S1, S2, S3, or a combination thereof, having a protein content of about 18% to about 25% by weight, a fat content of about 48% to about 56% by weight, and/or an L value of greater than 60 as measured by Hunter Colorflex color meter in its seeds, and comprising one or more introgressed shatter resistant capsule loci that are associated with a plurality of quantitative trait loci (QTLs); wherein said plurality of QTLs comprises QTL 1, QTL 2, QTL 3, QTL 4, QTL 5, QTL 6, and QTL 7; wherein the QTL 1 comprises the nucleic acid marker of SEQ ID NO: 1 or 9, the QTL 2 comprises the nucleic acid marker SEQ ID NO: 2 or 10, the QTL 3 comprises the nucleic acid marker of SEQ ID NO: 3 or 11, the QTL4 comprises SEQ ID NO: 4 or 12, the QTL 5 comprises the nucleic acid marker of SEQ ID NO: 5 or 13, the QTL 6 comprises the nucleic acid markers of SEQ ID NO: 6 or 14 and SEQ ID NO: 7 or 15, and the QTL 7 comprises the nucleic acid marker of SEQ ID NO: 8 or 16; wherein said nucleic acid markers are arranged in a marker cassette; and wherein said nucleic acid markers are associated with shatter resistance capsules; b) crossing the sesame plant having shatter resistance capsule of part a) with another sesame plant to produce F1 seeds; c) growing progeny plants from the F1 seeds; and d) selecting progeny sesame plants comprising the shatter resistant capsule loci and exhibiting shatter resistance capsule phenotype.

11. The method of claim 10, further comprising genotyping the progeny sesame plants for the presence of one or more of said nucleic acid markers associated with said QTLs.

12. The method of claim 11, wherein said genotyping comprises detecting said nucleic acid markers.

13. The method of claim 11, wherein the alleles of said nucleic acid markers are homozygous or heterozygous.

14. The method of claim 10, wherein the alleles for the nucleic acid markers SEQ ID NO: 9, 13, and 7 are homozygous or heterozygous.

15. The method of claim 10, wherein the alleles for the nucleic acid markers SEQ ID NO: 5, 11, and 16 are homozygous or heterozygous.

16. The method of claim 10, wherein said shatter resistant capsules comprise are a fully or partly a partial shatter resistant capsules.

17. The sesame plant or the part thereof according to claim 1, comprising Marker Cassette S,
wherein said Marker Cassette S comprises LG6_19788548, LG6_6028959, LG8_18013656, or a combination thereof, wherein the alleles for said LG6_19788548, LG6_6028959, and LG8_18013656 are homozygous or heterozygous;
wherein the nucleic acid sequence of LG6_19788548 is set forth in SEQ ID NO: 17 or 18;
wherein the nucleic acid sequence of LG6_6028959 is set forth in SEQ ID NO: 19 or 20; and
wherein the nucleic acid sequence of LG8_18013656 is set forth in SEQ ID NO: 21 or 22.

18. The method according to claim 10, wherein the sesame plant comprises Marker Cassette S,
wherein said Marker Cassette S comprises LG6_19788548, LG6_6028959, LG8_18013656, or a combination thereof, wherein the alleles for said LG6_19788548, LG6_6028959, and LG8_18013656 are homozygous or heterozygous;
wherein the nucleic acid sequence of LG6_19788548 is set forth in SEQ ID NO: 17 or 18;
wherein the nucleic acid sequence of LG6_6028959 is set forth in SEQ ID NO: 19 or 20; and
wherein the nucleic acid sequence of LG8_18013656 is set forth in SEQ ID NO: 21 or 22.

19. A method of making tahini, comprising
testing a protein content, a fat content and/or an L value of sesame seeds,
selecting sesame seeds having about 18% to about 25% protein content by weight, about 48% to about 56% fat content by weight, and/or an L value of greater than 60, as measured by Hunter Colorflex color meter; and
admixing the sesame seeds with ingredients.


SEQUENCE LISTING (22 sequences)

SEQ ID NO: 1 - DNA, 301 bp, Sesamum indicum
cctttagcagggcatcactctcttcaacatcgtactgcaccgagaggaatttcgaagtgagaaaaacttggggctccgacacttcccctttgctgtcgttttgaaaagtgagcttcaatttcgccaaattatccaacaatttgcacgagactttattaaagaacacggagctctcgctgtcgaactccgacgtaactcgcagcttcggccgacccatttggaagagccccagaaaaccgccgcctttcgaaccgccgtcgtccacggcgggggatggtggtggagacagataatagggttg

SEQ ID NO: 2 - DNA, 301 bp, Sesamum indicum
ccgggccacaactgatgcttattgtgttgccaaatatggtcagaagtgggtcaggacaaggacaatcatcgacagttttgctcctaaatggaatgagcagtatacttgggaagtttttgatccttgcactgttgtcaccattggtgtattcgataattgtcatctgcaaggtggagataaagctggaagggattcaagaattgggaaggtaagaattcgcctttcaactctggaaacagaccgtgtgtacactcattcttatcctcttctagttttgcatccttccggggtaaaaaagatg

SEQ ID NO: 3 - DNA, 301 bp, Sesamum indicum
ttagggcgtcgaattacttggacgcgccgagcaagctagcctgctgcaactcaagctcgttgagctgctgctcgcggtccatctctataatcagaggcacgacgagcacgaggaaggtggtgccggcaatccacgcggcttttccggtgctcttcaggagcttctttgcgacgtaggcggtatcaaaagccgctttcttgccgcggtacacgatgggtgactgggaaacggaggtggaaacacgggacaggattccgtcgtccgacgacgctccgcctcctctagtagacattctggtgaa

SEQ ID NO: 4 - DNA, 301 bp, Sesamum indicum
gccattgatttaccaaaagcaccaacccattttggtgagataattgggaaacttgttttggctggagccttggacttcaacaaggtggcaagggatattctggcaaaagtaggtgacgactattaccaaaaggccatatttactgctgctctgaaggttgtcagctctgatccttcaggaaaggcattgctcgattcacaggcgtctgatgtcgctgcctgcgagagtttattttagagctcactccttgttatgggaattactggaaacatttgtaacctcatagaagaaatgtgctatt

SEQ ID NO: 5 - DNA, 301 bp, Sesamum indicum
aatagaaaattggtaattcaaaagggaaagatggaaaaatgattaccatccctgtcttcagctgcctcaacattcacagaatccttcgcaagcacatctgtctctgtggatgctttactttttattaccggaaactgaacattgtgccgtccatttttcctaaaaagcatataatctctctttgacagtgttttcattacaagagttcctgcatttccatcattctaagaaagagaggttgattaaggcatccagcatcgcataaacataaccaggaaaatcgggagcaaacatagtactg

SEQ ID NO: 6 - DNA, 301 bp, Sesamum indicum
ggaggcaaaagaatacgggttggttgatgcagtgatcgatgatggcaagcctggactagtcgcacccatcgcagatactgcacccccaccaaaaacccgtgtctgggatctttggaaaatcgaaggcagtaaaaaagccaagaaaaacttgccctccgaagagaaactattacaaaatggatacacagttggccaaggtgaagatgacagaagcacggaacaggtagaggaagcaccaacatctcaatgagtaatgaatgttgagatatttcttgtatacactgtcaaacattgtagctag

SEQ ID NO: 7 - DNA, 301 bp, Sesamum indicum
agttgataaactgttgactaatcaaataatacgcattctgcacgcactcacaaatactatgattgttgtttactgaataaggttttcatggaattttcacaggttaaattctagtaatcacataaaagtatgtcgccagctgactcttcatgcgaggaaaatgtgtacatggccaagttggccgaacaggctgagaggtatgaggagatggttgaattcatggagaaggttgtgaaggccgtggacactgatgagctgacagtcgaggaaaggaaccttctctctgtggcatacaagaatg

SEQ ID NO: 8 - DNA, 301 bp, Sesamum indicum
acgtaaatgtttggatttaatgtaatttaatctaatgatattgcaaatgagtaaattactcccaaattattcggataaagcaatttaacttttggtttcttgtgagataacattgcatgtcctttatgaaccaagagcagtggccgagggactggtggtggtaccgttgccaaggatgcattaggcaatgatgttattgcagcggaatggctcaaaaaccatggacctggcgatcggacacttacacaggggctgaaggtaattattgatctagttgcaaaatagatcacttattggcttt

SEQ ID NO: 9 - DNA, 301 bp, Sesamum indicum
cctttagcagggcatcactctcttcaacatcgtactgcaccgagaggaatttcgaagtgagaaaaacttggggctccgacacttcccctttgctgtcgttttgaaaagtgagcttcaatttcgccaaattatccaacaatttgcacgagattttattaaagaacacggagctctcgctgtcgaactccgacgtaactcgcagcttcggccgacccatttggaagagccccagaaaaccgccgcctttcgaaccgccgtcgtccacggcgggggatggtggtggagacagataatagggttg

SEQ ID NO: 10 - DNA, 301 bp, Sesamum indicum
ccgggccacaactgatgcttattgtgttgccaaatatggtcagaagtgggtcaggacaaggacaatcatcgacagttttgctcctaaatggaatgagcagtatacttgggaagtttttgatccttgcactgttgtcaccattggtgtatttgataattgtcatctgcaaggtggagataaagctggaagggattcaagaattgggaaggtaagaattcgcctttcaactctggaaacagaccgtgtgtacactcattcttatcctcttctagttttgcatccttccggggtaaaaaagatg

SEQ ID NO: 11 - DNA, 301 bp, Sesamum indicum
ttagggcgtcgaattacttggacgcgccgagcaagctagcctgctgcaactcaagctcgttgagctgctgctcgcggtccatctctataatcagaggcacgacgagcacgaggaaggtggtgccggcaatccacgcggcttttccggtgcccttcaggagcttctttgcgacgtaggcggtatcaaaagccgctttcttgccgcggtacacgatgggtgactgggaaacggaggtggaaacacgggacaggattccgtcgtccgacgacgctccgcctcctctagtagacattctggtgaa

SEQ ID NO: 12 - DNA, 301 bp, Sesamum indicum
gccattgatttaccaaaagcaccaacccattttggtgagataattgggaaacttgttttggctggagccttggacttcaacaaggtggcaagggatattctggcaaaagtaggtgacgactattaccaaaaggccatatttactgctgctgtgaaggttgtcagctctgatccttcaggaaaggcattgctcgattcacaggcgtctgatgtcgctgcctgcgagagtttattttagagctcactccttgttatgggaattactggaaacatttgtaacctcatagaagaaatgtgctatt

SEQ ID NO: 13 - DNA, 301 bp, Sesamum indicum
aatagaaaattggtaattcaaaagggaaagatggaaaaatgattaccatccctgtcttcagctgcctcaacattcacagaatccttcgcaagcacatctgtctctgtggatgctttactttttattaccggaaactgaacattgtgccgtgcatttttcctaaaaagcatataatctctctttgacagtgttttcattacaagagttcctgcatttccatcattctaagaaagagaggttgattaaggcatccagcatcgcataaacataaccaggaaaatcgggagcaaacatagtactg

SEQ ID NO: 14 - DNA, 301 bp, Sesamum indicum
ggaggcaaaagaatacgggttggttgatgcagtgatcgatgatggcaagcctggactagtcgcacccatcgcagatactgcacccccaccaaaaacccgtgtctgggatctttggaaaatcgaaggcagtaaaaaagccaagaaaaacttaccctccgaagagaaactattacaaaatggatacacagttggccaaggtgaagatgacagaagcacggaacaggtagaggaagcaccaacatctcaatgagtaatgaatgttgagatatttcttgtatacactgtcaaacattgtagctag

SEQ ID NO: 15 - DNA, 301 bp, Sesamum indicum
agttgataaactgttgactaatcaaataatacgcattctgcacgcactcacaaatactatgattgttgtttactgaataaggttttcatggaattttcacaggttaaattctagtaatcacataaaagtatgtcgccagctgactcttcacgcgaggaaaatgtgtacatggccaagttggccgaacaggctgagaggtatgaggagatggttgaattcatggagaaggttgtgaaggccgtggacactgatgagctgacagtcgaggaaaggaaccttctctctgtggcatacaagaatg

SEQ ID NO: 16 - DNA, 301 bp, Sesamum indicum
acgtaaatgtttggatttaatgtaatttaatctaatgatattgcaaatgagtaaattactcccaaattattcggataaagcaatttaacttttggtttcttgtgagataacattgcatgtcctttatgaaccaagagcagtggccgaggggctggtggtggtaccgttgccaaggatgcattaggcaatgatgttattgcagcggaatggctcaaaaaccatggacctggcgatcggacacttacacaggggctgaaggtaattattgatctagttgcaaaatagatcacttattggcttt

SEQ ID NO: 17 - DNA, 198 bp, Sesamum indicum
actctatttggagtaaagattgagcaaatcgagagagtcagtacattcccagcattaacctgagagttaaaaataagcaaagtacctcatgaatacttctattttattcttttttcacccaagcataaacggaaaacactggaactgcaaagtctttcggacatgctaggtactgctgattcaccatcatcccatggc

SEQ ID NO: 18 - DNA, 198 bp, Sesamum indicum
actctatttggagtaaagattgagcaaatcgagagagtcagtacattcccagcattaacctgagagttaaaaataagcaaagtacctcatgaatacttttattttattcttttttcacccaagcataaacggaaaacactggaactgcaaagtctttcggacatgctaggtactgctgattcaccatcatcccatggc

SEQ ID NO: 19 - DNA, 301 bp, Sesamum indicum
gtcaaatttagaatacaaactcttcttcagttgttgctccatttggtaatcctttatctttaacagcttttaaagcttctgctgttgagtcgctgatatccagtagatactgtattctggccaccttatcagctgctgggtcatttttcaggtatatgaggaacagatcagccagttcctcgggcacctcccatgacaatggggttgagggtacggctttatcgcaggctaacaagtcattgagagaattaacctacaagattgtacaaaggcaactgagtaatgtgtttaagataaatga

SEQ ID NO: 20 - DNA, 301 bp, Sesamum indicum
gtcaaatttagaatacaaactcttcttcagttgttgctccatttggtaatcctttatctttaacagcttttaaagcttctgctgttgagtcgctgatatccagtagatactgtattctggccaccttatcagctgctgggtcatttttcatgtatatgaggaacagatcagccagttcctcgggcacctcccatgacaatggggttgagggtacggctttatcgcaggctaacaagtcattgagagaattaacctacaagattgtacaaaggcaactgagtaatgtgtttaagataaatga

SEQ ID NO: 21 - DNA, 301 bp, Sesamum indicum
ttggaggaaaaaattgaaataaataaagaaaacccagatcatcaaaccatgaacgataaagaactgaagctaaacaaacttacggccgcagttgctaatggaaggtgcagaaacatggcaggcagcatgcagccccaacgccttgctcacgttcagagtgacccccaactgcgcgctgttcttaaacgcctcgcaaaggcactccgcgtcggttttgagcacagtcttgagccccgaacagcacgtgccctccggcttcttcgacgtgcttcccgccgtcacgtaagacaaacagtccgcc

SEQ ID NO: 22 - DNA, 301 bp, Sesamum indicum
ttggaggaaaaaattgaaataaataaagaaaacccagatcatcaaaccatgaacgataaagaactgaagctaaacaaacttacggccgcagttgctaatggaaggtgcagaaacatggcaggcagcatgcagccccaacgccttgctcacattcagagtgacccccaactgcgcgctgttcttaaacgcctcgcaaaggcactccgcgtcggttttgagcacagtcttgagccccgaacagcacgtgccctccggcttcttcgacgtgcttcccgccgtcacgtaagacaaacagtccgcc